(19) United States
(12) Patent Application Publication
(10) Pub. No.: US 2021/0332411 A1
(43) Pub. Date: Oct. 28, 2021

(54) METHODS AND REAGENT FOR ANALYSING NUCLEIC ACIDS FROM INDIVIDUAL CELLS

(71) Applicant: AUTOLUS LIMITED, London (GB)

(72) Inventors: Biao Ma, London (GB); Leo Kassimatis, London (GB); Rosalind Gealy, London (GB); Yuchen Bai, London (GB); Shimobi Onuoha, London (GB); Martin Pulé, London (GB)

(21) Appl. No.: 17/284,147

(22) PCT Filed: Oct. 10, 2019
(86) PCT No.: PCT/GB2019/052881

§ 371 Date: Apr. 9, 2021

(30) Foreign Application Priority Data
Oct. 10, 2018 (GB) 1816520.9

(51) Int. Cl. C12Q 1/6804; C07K 16/28; C12Q 1/6806; C12Q 1/6869; C07K 16/42

(52) U.S. Cl. CPC C12Q 1/6804; C07K 16/2896; C12Q 1/6806; C07K 2317/522; C07K 16/4283; C07K 2317/31; C07K 2317/569; C12Q 1/6869

(57) ABSTRACT

The present disclosure relates to a bispecific reagent and a detection agent which are useful for the identification of an antibody-producing cell which produces an antibody that binds specifically to a target antigen. Further, the present disclosure relates to an assay for the identification of an antibody-producing cell which produces an antibody that binds specifically to a target antigen. The present disclosure also relates to kits comprising the bispecific reagent and the detection agent.

### FIELD OF THE INVENTION

The present invention relates to reagents and assays for identifying an antibody-producing cell which produces an antibody which binds to a target antigen.

### BACKGROUND TO THE INVENTION

The ‘age of monoclonal antibodies’ was ushered in with the advent of hybridoma technology in the 1970s. In the following decades, a myriad of monoclonal antibodies (mAbs) have been discovered and applied as reagents, diagnostics and therapeutics. The therapeutic market exceeded USD 75 billion in 2014, and there are more than 300 antibody products in development. The compounded annual growth rate of the mAb market is 8% (Ecker et al., 2015, MAbs 7:9-14). Throughout this period of rapid growth, antibody discovery has relied heavily on classical hybridoma technology in spite of three significant limitations. The first is the potential four-log loss of repertoire during the cellular fusion process that vastly reduces the likelihood of finding mAbs with rare attributes. Second, targets that are highly conserved between mammals are often poorly immunogenic in rodents. Finally, it is generally difficult using hybridoma technology to raise antibodies that are rodent cross-reactive, a necessary property for testing in many animal models of disease.

To address these limitations, numerous alternative antibody discovery approaches have been developed, most notably phage and ribosome display and more recently yeast and mammalian cell display. Antibody libraries have been made with either natural or synthetic antibody sequences, including human-derived sequences. While some naive libraries can be very large in size, with sizes estimated at over 10¹¹ members, antibodies discovered from these sources often require additional in vitro affinity maturation to reach potency comparable to traditional hybridoma antibodies. In some cases, phage libraries have been made with affinity-matured sequences from immunised animals. However, the original heavy and light chain pairings found in the B cells are generally lost in the cloning process, resulting in diminished diversity of antigen-specific clones and, in the absence of in vivo affinity maturation, an increased frequency of off-target reactivity.

In order to avoid traditional hybridoma fusion and combinatorial display, many methods that enable the sampling of memory B cell subsets have been developed, but few allow for the direct interrogation of the plasma cell repertoire, i.e. the subset of B cells responsible for producing immunoglobulin in serum. These methods have in common the need for single cell isolation. Traditional methods for single-cell isolation are micromanipulation, fluorescent-activated cell sorting (FACS), and laser capture microdissection (LCM). The throughput of the methods is also quite different. While FACS is efficient in cell sorting, micromanipulation and LCM are very slow and laborious techniques, allowing only very limited throughput. Once isolated, the single cells are then processed to obtain their genetic information.

By using the gel encapsulated microenvironment (GEM) assay, Mettler Izquierdo et al. were able to sample the B cell repertoire by analysing single antibody-secreting cells in a droplet containing multiple particulate reporters by microscopy (Mettler Izquierdo et al., 2016, Microscopy 65:341-352). Typically, at least two types of reporters are used to confirm specificity. GEMs are viewed on the microscope, manually harvested and the V regions of the heavy and light chains are obtained by RT-PCR.

Another approach is the so-called fluorescent foci method developed by Clargo and colleagues (Clargo et al., 2014, mAbs 6:143-59), which consists in mixing IgG-secreting cells, a source of solid phase antigen and a fluorescent-labelled anti-Fey-specific secondary reagent, and plating the mix out as a monolayer on a glass slide. Single specific B cells are visualised by fluorescence microscopy and subsequently isolated using a micromanipulator device for variable region recovery by RT-PCR.

However, these technologies are tremendously slow and tedious and require manual manipulation, which can lead to missing significant leads. Accordingly, there is a need in the art for an improved method for the direct interrogation of the plasma cell repertoire.

### SUMMARY OF ASPECTS OF THE INVENTION

The inventors have developed reagents and methods to characterise the genetic information encoded by individual antibody-secreting cells in a swift and straightforward manner. The present invention is particularly advantageous because it enables the direct acquisition and characterisation of the nucleotide sequences encoding the variable domains of antibodies with the desired antigen-binding characteristics (e.g. specificity, affinity) in a manner which is susceptible to automation. Key advantages include that a large number of cells is prepared in a relatively short time, thereby reducing the risk of missing leads, the cost of analysis per cell is low, and the methods provide an end-to-end solution.

Thus, in a first aspect, the invention provides a bispecific reagent which comprises:
(i) a first domain which binds specifically to an antigen expressed by an antibody-producing cell; and
(ii) a second domain which comprises a target antigen.

The antigen expressed by the antibody-producing cell may be selected from CD138, CD38, CD98, Sca-1, Ly6c1/2, Ly6k, CD28, transmembrane activator and calcium modulator and cyclophilin ligand interactor (TACI), B-cell maturation antigen (BCMA), and SLAM7.

The first domain may be selected from a monoclonal antibody, a fragment thereof, and a domain antibody (dAb).

In a second aspect, the present invention provides a complex C1, which comprises:
a) an antibody-producing cell; and
b) a bispecific reagent according to the first aspect of the invention, which is bound to the antibody-producing cell via the antigen expressed by the antibody-producing cell.

In a third aspect, the present invention provides a complex C2, which comprises:
a) an antibody-producing cell;
b) a bispecific reagent according to the first aspect of the invention, bound to the antibody-producing cell via the antigen expressed by the antibody-producing cell; and
c) an antibody which:
   (i) is secreted by the antibody-producing cell, and
   (ii) specifically binds the target antigen,
      wherein the antibody is bound to the bispecific reagent via the target antigen.

In a fourth aspect, the present invention provides a detection agent which comprises an anti-IgG antibody which is labelled with a nucleic acid which comprises a specific tag sequence.

The anti-IgG antibody may be a scFv or a nanobody.

The nanobody may be selected from Alpaca-anti-Mouse IgG1 monoclonal nanobody TP1104, Alpaca-anti-Mouse IgG2a monoclonal nanobody TP1129, Alpaca-anti-Mouse IgG2a/2b monoclonal nanobody TP925, Alpaca-anti-Mouse IgG3 monoclonal nanobody TP924, and alpaca-anti-mouse IgG2a Fc monoclonal nanobody TP923.

The nucleic acid which comprises a specific tag sequence may be DNA.

The nucleic acid which comprises a specific tag sequence further may comprise a sequence suitable for priming in next generation sequencing (NG sequencing).

The nucleic acid which comprises a specific tag sequence may further comprise a sequence that is complementary to that of a template switching oligonucleotide.

The nucleic acid may comprise the sequence shown as SEQ ID NO: 8.

The nucleic acid which may comprise a specific tag sequence is RNA.

The nucleic acid which comprises a specific tag sequence may further comprise a specific tag sequence and a Poly(A) tail.

The RNA sequence may encode the anti-IgG antibody.

The anti-IgG antibody and the RNA sequence may be linked together via a puromycin.

The RNA sequence may comprise an RNA analogue.

The RNA sequence may be at least 450 nucleotides long.

In a fifth aspect, the present invention provides a complex C3, which comprises:
a) an antibody-producing cell;
b) a bispecific reagent according to the first aspect of the invention, bound to the antibody-producing cell via the antigen expressed by the antibody-producing cell;
c) an antibody which:
   (i) is secreted by the antibody-producing cell, and
   (ii) specifically binds the target antigen, wherein the antibody is bound to the bispecific reagent via the target antigen; and
d) a detection agent according to the fourth aspect of the invention, bound to the antibody of (c).

In a sixth aspect, the present invention provides an assay for identifying an antibody-producing cell which produces an antibody which binds specifically to a target antigen, which comprises the following steps:
(i) providing a population of antibody-producing cells;
(ii) binding a bispecific reagent according to the first aspect of the invention to the cells;
(iii) incubating the cells from step (ii) with a target antigen;
(iv) adding a detection agent according to the fourth aspect of the invention to the cells from step (iii);
(v) partitioning the cells from (iv) into droplets, wherein each droplet contains a single cell and a unique barcode molecule;
(vi) performing reverse transcription such that all RNA molecules in the cell within the partition and the RNA sequence of the detection reagent (if present) are barcoded with the unique barcode molecule;
(vii) disrupting the partitions and pooling the barcoded nucleic acid sequences from (vi)
(viii) analysing the pooled sequences to find sets of sequences with the same unique barcode which comprise:
   (a) a sequence encoding a heavy chain variable domain (VH);
   (b) a sequence encoding a light chain variable domain (VL); and
   (c) a sequence corresponding to the reverse transcript of the RNA sequence of the detection agent

The assay may comprise a step of sorting cells prior to step (i).

The reverse transcription may be performed using an oligonucleotide which is complementary to a sequence encoding the IgG heavy chain constant region.

The reverse transcription may be performed using an oligonucleotide which is complementary to a sequence encoding the IgG light chain constant region.

The assay may comprise a step of DNA amplification that is performed after step after step (vi) and prior to step (vii).

The assay may comprise a step of DNA amplification is performed after step after step (vii) and prior step (viii).

The analysis of step (viii) may comprise a step of DNA sequencing.

In a seventh aspect, the present invention provides a kit for use in the assay of the sixth aspect of the invention, which comprises the bispecific reagent according to the first aspect of the invention and the bispecific reagent according to the fourth aspect of the invention.

The kit may comprise one or more components selected from the group consisting of partitioning fluids, barcode molecule libraries, which may be associated or not with beads (e.g. microcapsules), reagents for disrupting cells, reagents for amplifying nucleic acids, and any other component required to carry out the assay of the invention.

The kit may comprise instructions for using the kit according to the assay of the invention.

In an eighth aspect, the present invention provides a bispecific reagent which comprises:
(i) a first domain which binds specifically to an antigen expressed by an antibody-producing cell; and
(ii) a second domain which comprises a binding domain which binds specifically to IgG.

The antigen expressed by the antibody-producing cell may be selected from CD138, CD38, CD98, Sca-1, Ly6c1/2, Ly6k, CD28, transmembrane activator and calcium modulator and cyclophilin ligand interactor (TACI), B-cell maturation antigen (BCMA), and SLAM7.

The first domain may be selected from a monoclonal antibody, a fragment thereof, and a domain antibody (dAb).

The nanobody may be selected from Alpaca-anti-Mouse IgG1 monoclonal nanobody TP1104, Alpaca-anti-Mouse IgG2a monoclonal nanobody TP1129, Alpaca-anti-Mouse IgG2a/2b monoclonal nanobody TP925, Alpaca-anti-Mouse IgG3 monoclonal nanobody TP924, and alpaca-anti-mouse IgG2a Fc monoclonal nanobody TP923.

In a ninth aspect, the present invention provides a complex D1 which comprises:
a) an antibody-producing cell; and
b) a bispecific reagent according to the eighth aspect of the invention, which is bound to the antibody-producing cell via the antigen expressed by the antibody-producing cell.

In a tenth aspect, the present invention provides a complex D2 which comprises:
a) an antibody-producing cell; and
b) a bispecific reagent according to the eighth aspect of the invention, which is bound to the antibody-producing cell via the antigen expressed by the antibody-producing cell; and
c) an antibody which:
   (i) is secreted by the antibody-producing cell, and
   (ii) specifically binds the target antigen,
   wherein the antibody is bound to the bispecific reagent via the binding domain which binds specifically to IgG.

In an eleventh aspect, the present invention provides a detection agent which comprises the target antigen which is labelled with a nucleic acid which comprises a specific tag sequence.

The nucleic acid which may comprise a specific tag sequence is DNA.

The nucleic acid which may comprise a specific tag sequence further comprises a sequence suitable for priming in NG sequencing.

The nucleic acid which comprises a specific tag sequence may further comprise a sequence that is complementary to that of a template switching oligonucleotide.

The nucleic acid may comprise the sequence shown as SEQ ID NO: 8.

The nucleic acid which comprises a specific tag sequence may be RNA.

The nucleic acid which comprises a specific tag sequence may further comprise a specific tag sequence and a Poly(A) tail.

The RNA sequence may encode the target antigen.

The target antigen and the RNA sequence may be linked together via a puromycin.

The RNA sequence may be at least 450 nucleotides long.

In a twelfth aspect, the present invention provides a complex D3 which comprises:
a) an antibody-producing cell; and
b) a bispecific reagent according to the eighth aspect of the invention, which is bound to the antibody-producing cell via the antigen expressed by the antibody-producing cell; and
c) an antibody which:
   (i) is secreted by the antibody-producing cell, and
   (ii) specifically binds the target antigen,
wherein the antibody is bound to the bispecific reagent via the binding domain which binds specifically to IgG; and
d) a detection agent according to the eleventh aspect of the invention, which bound to the antibody of (c).

In a thirteenth aspect, the present invention provides an assay for identifying an antibody-producing cell which produces an antibody which binds to a target antigen, which comprises the following steps:
(i) providing a population of antibody-producing cells;
(ii) binding a bispecific reagent according to the eighth aspect of the invention to the cells;
(iii) incubating the cells from step (ii) with a target antigen;
(iv) adding a detection agent according to the eleventh aspect of the invention to the cells from step (iii);
(v) partitioning the cells from (iv) into partitions, wherein each partition contains a single cell and a unique barcode molecule;
(vi) performing reverse transcription such that all RNA molecules in the cell within the partition and the RNA sequence of the detection reagent (if present) are barcoded with the unique barcode molecule;
(vii) disrupting the partitions and pooling the barcoded nucleic acid sequences from (vi)
(viii) analysing the pooled sequences to find sets of sequences with the same unique barcode which comprise:
   (a) a sequence encoding a heavy chain variable domain (VH);
   (b) a sequence encoding a light chain variable domain (VL); and
   (c) a sequence corresponding to the reverse transcript of the RNA sequence of the detection agent.

The assay may comprise a step of sorting cells prior to step (i).

The reverse transcription may be performed using an oligonucleotide which is complementary to a sequence encoding the IgG heavy chain constant region.

The reverse transcription may be performed using an oligonucleotide which is complementary to a sequence encoding the IgG light chain constant region.

The assay may comprise a step of DNA amplification is performed after step after step (vi) and prior to step (vii).

The assay may comprise a step of DNA amplification is performed after step after step (vii) and prior step (viii).

In a particular embodiment, the analysis of step (viii) comprises a step of DNA sequencing.

In a fourteenth aspect, the present invention provides a kit for use in the assay according to thirteenth aspect of the invention, which comprises the bispecific reagent according to the eighth aspect of the invention and the detection agent according to the eleventh aspect of the invention.

The kit may comprise one or more components selected from the group consisting of partitioning fluids, barcode molecule libraries, which may be associated or not with beads (e.g. microcapsules), reagents for disrupting cells, reagents for amplifying nucleic acids, and any other component required to carry out the assay of the invention.

The kit according may comprise instructions for using the kit according to the assay according to the fourteenth aspect of the invention.

### DESCRIPTION OF THE FIGURES

FIG. 1. Schematic representation depicting the method of processing antibody-producing cells obtained from vaccinated animals for the assessment of production of antibodies that specifically bind a target antigen.

FIG. 2. Schematic representation showing the method of processing the RNA transcripts of each individual antibody-producing cell to obtain barcoded cDNA molecules for subsequent in vitro analysis.

FIG. 3. Diagrams showing the cell lines and reagents developed. A) Two cell lines bearing the characteristics of Mouse plasma cells. The surface expression of Mouse CD138, and secretion of Mouse-anti-Human BCMA or TACI monoclonal antibody are indicated. Jurkat cells express a functional Human TCRα, β, and Sup-T1 cells do not. The makeup of retro-viral trans-gene in either cell line is shown (2A, self-cleaving 2A peptide; HC, IgG heavy chain; LC, Igκ). B) Proposed structure of bispecific reagents 63424 (left) and 63425 (right). C) Secondary antibody TP923 derived from an alpaca-anti-Mouse IgG2a Fc nanobody (in orange, labelled as VHH) and conjugated with an oligonucleotide containing a specific sequence tag. The nanobody was expressed as a heavy chain only antibody by fusion with Human IgG1 hinge, CH2 and CH3. The sequence of the oligonucleotide is shown as SEQ ID NO: 8 and the particular modifications of are annotated (5AmMC6, 5′ amino modified C6; Read 2N, Illumina sequencing priming site; *, Phosphorothioate Bond).

FIG. 4. Analysis of the cell lines and reagents developed by flow cytometry. Jurkat.MuCD138.aBCMA cells which secrete mouse-anti-human BCMA 5G10 monoclonal antibody (MAb) (left) and Sup-T1.MuCD138.aTACI cells (right) which secrete mouse-anti-human TACI 4G9 MAb were incubated with bispecific reagents 63424 or 63425, containing the extracellular domain of human BCMA or human TACI, respectively, followed by stained with PE-F(ab′)₂-Goat anti-Mouse IgG-Fc secondary antibody. Single parameter histograms are shown where the values on the x-axis indicate the fluorescence intensity of PE, and the values on the y-axis indicate the cell counts.

FIG. 5. Diagrams showing the barcoded nucleic acids resulting from the reverse transcription in each Gel bead in EMulsion (GEM). A) cDNA molecule containing the sequencing adapter, a unique molecular identifier (UMI), and a shared 10× barcode per GEM at its 5′ end. B) The reverse transcription product resulting from the DNA sequence from the oligonucleotide conjugated to secondary antibodies contain the sequencing adapter, a UMI, a shared 10× barcode per GEM, the specific tag, and a Read 2N sequence for next generation sequencing.

### DETAILED DESCRIPTION OF THE INVENTION

The inventors have developed assays and reagents for identifying an antibody-producing cell which produces an antibody which binds specifically to a target antigen. The assay exploits the use of a bispecific reagent to capture the antibody by the same cell which produces it. This allows the retrieval of the mRNA encoding the variable regions of the antibody. Advantageously, the assay is susceptible to automation and does not require the manual manipulation of samples to isolate single antibody-secreting cells.

1. Bispecific Reagents

The present invention provides a bispecific reagent that is suitable for capturing an antibody which specifically binds a target antigen onto the antibody-secreting cell (ASC) which secreted it.

Thus, the present invention relates to a bispecific reagent, hereinafter “the bispecific reagent of the invention”, which comprises a first domain which binds specifically to an antigen expressed by an ASC; and a second domain which binds to an antibody.

In the context of the present invention, the terms antibody-secreting cell, ASC and antibody producing cell are used synonymously, and relate to B cells that have undergone differentiation into plasmablasts and plasma cells. ASCs may be of any mammalian species, although mouse, rat, rabbit, camelid, and human species are particularly preferred. The first and second domains may be linked by any suitable means, many of which are known in the art. Accordingly, the bispecific reagent of the invention may be, without limitation, a fusion protein or a protein conjugate.

A “fusion protein”, in the context of the present invention, refers to a protein generated through the joining of two or more genes that originally coded for separate proteins or protein domains. Typically, the proteins or protein domains are connected end-to-end via fusion of N- and/or C-termini between the proteins, or by the insertion of a protein linker. Protein linkers, or linkers, aid fusion protein design by providing appropriate spacing between domains, supporting correct protein folding. Linkers may be flexible, which may comprise glycine residues, or rigid, which may comprise proline residues. Examples of protein linkers are well known in the art.

A “protein conjugate”, in the context of the present invention, refers to proteins or protein domains that are covalently linked together by means of crosslinking agents. Crosslinking reagents are commercially available and have a wide range of characteristics, including functional group specificity, with reactive moieties that target amines, sulfhydryls, carboxyls, carbonyls or hydroxyls; homo or heterobifunctionality; and variable spacer arm length.

Non-limiting examples of chemical cross-linkers include cystamine, gluteraldehyde, dimethyl suberimidate, N-Hydroxysuccinimide crosslinker BS3, formaldehyde, carbodiimide (EDC), SMCC, Sulfo-SMCC, vinylsilane, N,N′diallyltartardiamide (DATD), N,N′Bis(acryloyl)cystamine (BAC), or homologues thereof.

The first domain which binds specifically to an antigen expressed by an ASC may be selected from a monoclonal antibody, a fragment thereof, and a domain antibody (dAb) or VHH or nanobody, or a ligand specific for said antigen expressed by an ASC. Where the first domain is a fragment of a monoclonal antibody, it may be selected from the group comprising, without limitation, a scFv and a Fab.

It will be appreciated that it is particularly advantageous that the antigen to which the first domain binds specifically is restricted in expression to ASCs. This will allow the bispecific reagent to recognise B cells that have undergone differentiation into plasmablasts and plasma cells, i.e. ASCs, via the first domain while the second domain is able to capture the antibody that the ASCs secrete. Thus, the antigen expressed by the ASC may be selected from CD138, CD38, CD98, Sca-1, Ly6c1/2, Ly6k, CD28, transmembrane activator and calcium modulator and cyclophilin ligand interactor (TACI), B-cell maturation antigen (BCMA), and SLAM7, which are antigens expressed by murine ASCs. In the event that ASCs from other species are utilised, the person skilled in the art will appreciate that the first domain will be specific to an antigen which has restricted expression in ASCs of the particular species.

The second domain which binds to an antibody may interact specifically with the antigen binding site or with the rest of the molecule. Thus, the second domain may comprise a target antigen or a binding domain which binds specifically to IgG.

The “target antigen”, in the context of the present invention, refers to an antigen of interest or an antigen against which there is a wish to generate novel antibodies. Virtually any target antigen may be used for the purposes of the invention. Examples of target antigens include, without limitation, CD19, CD20, CD21, CD22, CD33, CD38, CD45, CD52, CD79a, CD79b, CEA, GD2, BCMA, HER2, EGFR, PD-1, PD-L1, TACI, FcRH5, ROR1, DLL3.

The target antigen may be human or from a non-human species, such as mouse, rat, rabbit, dog, cat, cow, sheep, pig, and camelid. Preferably, the target antigen is human.

Where the second domain of the bispecific reagent comprises a binding domain which binds specifically to IgG, this may be selected from a monoclonal antibody, a fragment thereof, and a dAb or VHH or nanobody, or a receptor specific for IgG, such as members of the FcγR family, FcRn, and TRIM21. Where the binding domain which binds specifically to IgG is a fragment of a monoclonal antibody, it may be selected from the group comprising, without limitation, a F(ab′)₂, a Fab and a scFv. Non-limiting examples of commercially available IgG-specific antibodies include goat anti-mouse IgG Fc monoclonal antibody (abcam, Cat. No. ab197780), goat anti-mouse IgG Fc monoclonal antibody (ThermoFisher, Cat. No. SA5-10227), AffiniPure goat anti-mouse IgG (subclasses 1+2a+2b+3), Fc specific antibody (Jackson ImmunoResearch, Cat. No. 115-005-164), AffiniPure F(ab′)₂ fragment goat anti-mouse IgG, Fc specific (Jackson ImmunoResearch, Cat. No. 115-006-071), AffiniPure Fab fragment goat anti-mouse IgG1, Fc specific (Jackson ImmunoResearch, Cat. No. 115-007-185), AffiniPure Fab fragment goat anti-mouse IgG2a, Fc specific (Jackson ImmunoResearch, Cat. No. 115-007-186), and AffiniPure Fab fragment Goat anti Mouse IgG2b, Fc specific (Jackson ImmunoResearch, Cat. No. 115-007-187).

The terms “domain antibody”, “dAb”, VHH, and nanobody are used herein as synonyms.

2. Complexes C1 and D1

In another aspect, the present invention relates to a first complex, hereinafter “the complex C1 of the invention”, which comprises:
a) an antibody-producing cell; and
b) the bispecific reagent according to the invention, which is bound to the antibody-producing cell via the antigen expressed by the antibody-producing cell.

In another aspect, the present invention relates to a complex D1, hereinafter “the complex D1 of the invention”, which comprises:
a) an antibody-producing cell; and
b) the bispecific reagent according to the invention, which is bound to the antibody-producing cell via the antigen expressed by the antibody-producing cell.

The terms “antibody-producing cell” and “bispecific reagent” have been defined in the context of the bispecific reagent of the invention, and the particular features and embodiments described therein are equally applicable to the complexes C1 and D1 of the invention.

It will be immediately appreciated that the bispecific reagent will bind to the antibody-producing cell when both come into contact via the first domain of the bispecific reagent, which binds specifically to an antigen expressed by an antibody-producing cell. Once bound to the antibody-producing cell, the second domain of the bispecific reagent, which binds an antibody, is free to bind an antibody.

The antibody-producing cell may be a plasmablast, a plasma cell or a memory B cell.

The antibody-producing cell may be a murine, rat, rabbit, camelid, or human cell line.

3. Complexes C2 and D2

In another aspect, the present invention relates to a second complex, which comprises:
a) an antibody-producing cell;
b) a bispecific reagent according to the invention, bound to the antibody-producing cell via the antigen expressed by the antibody-producing cell; and
c) an antibody which:
   (i) is secreted by the antibody-producing cell, and
   (ii) specifically binds the target antigen,
   wherein the antibody is bound to the bispecific reagent via the second domain thereof.

Where the second domain of the bispecific reagent comprises a target antigen, the antibody secreted by the antibody-producing cell is bound specifically to the target antigen. Ideally, the binding will occur via the complementary-determining regions of the antibody.

Thus, the second complex, i.e. the complex C2, may comprise:
a) an antibody-producing cell;
b) a bispecific reagent according to the invention, bound to the antibody-producing cell via the antigen expressed by the antibody-producing cell; and
c) an antibody which:
   (i) is secreted by the antibody-producing cell, and
   (ii) specifically binds the target antigen,
   wherein the antibody is bound to the bispecific reagent via the target antigen

Alternatively, where the second domain of the bispecific reagent comprises a binding domain which binds specifically to IgG, the antibody secreted by the antibody-producing cell is bound specifically to this.

Thus, the second complex, i.e. the complex D2, may comprise:
a) an antibody-producing cell;
b) a bispecific reagent according to the invention, bound to the antibody-producing cell via the antigen expressed by the antibody-producing cell; and
c) an antibody which:
   (i) is secreted by the antibody-producing cell, and
   (ii) specifically binds the target antigen,
   wherein the antibody is bound to the bispecific reagent via the binding domain which binds specifically to IgG.

It will be readily appreciated that two of the elements of the complexes C2 and D2, i.e. the antibody-producing cell (a) and the bispecific reagent according to the invention (b), which is bound to the antibody-producing cell via the antigen expressed by the antibody-producing cell, constitute the complexes C1 and D1 of the invention. Accordingly, the definitions, particular features and embodiments described in the context of the complexes C1 and D1 of the invention are equally applicable to the complexes C2 and D2 of the invention.

The bispecific reagent is bound to the antibody-producing cell via the first domain which binds specifically to an antigen expressed by the antibody-producing cell. This leaves the second domain of the bispecific reagent, which binds to an antibody, is free to bind the antibody.

The complexes C2 and D2 additionally comprises a third element (c), i.e. an antibody which (i) is secreted by the antibody-producing cell, and which (ii) specifically binds target antigen.

4. Detection Agent

The present invention also relates to a detection agent which is suitable for the detection of an antibody which (i) is secreted by the antibody-producing cell, and (ii) specifically binds the second domain of the bispecific reagent, such as the one that forms part of a complex C2 and D2 according to the invention, wherein the antibody is bound to the bispecific reagent via the second domain thereof.

Thus, in another aspect, the present invention relates to a detection agent, hereinafter “the detection agent of the invention”, which comprises a moiety that is suitable for binding specifically an antibody and which is labelled with a nucleic acid which comprises a specific tag sequence.

The moiety that is suitable for binding specifically an antibody may be an anti-IgG antibody or the target antigen that is recognised by the antibody.

In an embodiment, the detection agent comprises an anti-IgG antibody which is labelled with a nucleic acid which comprises a specific tag sequence.

The anti-IgG antibody may be selected from a monoclonal antibody, a fragment thereof, and a dAb, VHH, or nanobody. Where the binding domain which binds specifically to IgG is a fragment of a monoclonal antibody, it may be selected from the group comprising, without limitation, a F(ab′)₂, a Fab and a scFv. Any antibody, fragment thereof, or VHH, nanobody or dAb that specifically binds an IgG may be used. Non-limiting examples of commercially available IgG-specific antibodies include goat anti-mouse IgG Fc monoclonal antibody (abcam, Cat. No. ab197780), goat anti-mouse IgG Fc monoclonal antibody (ThermoFisher, Cat. No. SA5-10227), AffiniPure goat anti-mouse IgG (subclasses 1+2a+2b+3), Fc specific antibody (Jackson ImmunoResearch, Cat. No. 115-005-164), AffiniPure F(ab′)₂ fragment goat anti-mouse IgG, Fc specific (Jackson ImmunoResearch, Cat. No. 115-006-071), AffiniPure Fab fragment goat anti-mouse IgG1, Fc specific (Jackson ImmunoResearch, Cat. No. 115-007-185), AffiniPure Fab fragment goat anti-mouse IgG2a, Fc specific (Jackson ImmunoResearch, Cat. No. 115-007-186), and AffiniPure Fab fragment Goat anti Mouse IgG2b, Fc specific (Jackson ImmunoResearch, Cat. No. 115-007-187).

The anti-IgG antibody may be a dAb, VHH or nanobody. The dAb, VHH or nanobody may be selected from alpaca-anti-mouse IgG1 monoclonal nanobody TP1104, alpaca-anti-mouse IgG2a monoclonal nanobody TP1129, alpaca-anti-mouse IgG2a/2b monoclonal nanobody TP925, alpaca-anti-mouse IgG3 monoclonal nanobody TP924, and alpaca-anti-mouse IgG2a Fc monoclonal nanobody TP923 as described by Pleiner et al. (J Cell Biol, 2018, 217: 1143-54).

In an embodiment, the moiety that is suitable for binding specifically an antibody is the alpaca-anti-mouse IgG2a Fc monoclonal nanobody TP923.

The detection agent is labelled with a nucleic acid comprises a specific tag sequence.

The nucleic acid may be DNA or RNA.

The overall nucleic acid is coupled to anti-IgG antibody by any suitable linkage, such as a disulfide linker or the linkage obtained with the Thunder-Link® PLUS Oligo Conjugation System (Expedeon), which requires an aminated oligonucleotide, preferably a 5′ aminated oligonucleotide. Any other coupling chemistry may be used for the purposes of this invention.

In order to identify the antibody which (i) is secreted by the antibody-producing cell, and (ii) specifically binds the second domain of the bispecific reagent as described above, the nucleic acid of the detection agent of the invention contains a specific tag sequence or labelling sequence. The term “tag sequence” or “labelling sequence”, as used herein, refers to any sequence that serves to identify such an antibody. It may be an arbitrary sequence.

The specific tag sequence provides a unique sequence segment (USS), e.g. a random sequence, such as a random N-mer sequence. This unique sequence serves to provide a unique identifier of the antibody, which (i) is secreted by the antibody-producing cell, and (ii) specifically binds the target antigen, that was captured onto the antibody-producing cells that secreted it by the bispecific reagent.

When used in the methods according to the invention, the USS provides a label on individual antibody-producing cells which secrete antibodies that are specific to an antigen of interest. The label is reversed transcribed and incorporated into the pool of cDNA originating from said cell thus readily allowing the identification of the cell producing an antibody specific to an antigen of interest in a population containing antibody-producing cells.

This unique sequence segment (USS) may include from 5 to about 500 or more nucleotides within the sequence of oligonucleotides. The USS sequence segment can be at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, at least 500 nucleotides in length or longer.

The USS may consist of between 5 and 500 nucleotides, or between 10 and 400 nucleotides, or between 15 and 300 nucleotides, or between 20 and 200 nucleotides, or between 30 and 100 nucleotides, or between 40 and 90 nucleotides, or between 50 and 80 nucleotides in length.

Examples of USS include, without limitation, the following sequences:

(SEQ ID NO: 5)
5′-ACGTGACTACACGAATCAATCTGTGCTAGACTGC-3′
(SEQ ID NO: 1)
5′-UCACCCCUCAACAACUAGCAAAGGCAGCCCCAUAAACACACAGUAUGU
UUUUUGA-3′
(SEQ ID NO: 2)
5′-UAGGAAAGUUGGUCUUCGCCAUCAUGGCAGUUGCUUGCAAUGUAA
UUUUCAGUUAA-3′

The specific tag sequence may include from 5 to about 500 or more nucleotides within the sequence of oligonucleotides. The USS sequence segment can be at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, at least 500 nucleotides in length or longer.

The specific tag sequence may consist of between 5 and 500 nucleotides, or between 10 and 400 nucleotides, or between 15 and 300 nucleotides, or between 20 and 200 nucleotides, or between 30 and 100 nucleotides, or between 40 and 90 nucleotides, or between 50 and 80 nucleotides in length.

The structure of the specific tag sequence may include a number of sequence elements in addition to the oligonucleotide barcode or specific tag sequence. These additional elements enable using the detection agent in the methods according to the invention. Examples of the additional elements include a sequence suitable for priming in methods of next generation sequencing, a sequence that is complementary to that of a template switching oligonucleotide, and, where the oligonucleotide is RNA, a poly-A tail. The specific tag sequence may contain one, two or more of these additional elements.

Thus, in an embodiment, the nucleic acid further comprises a sequence suitable for priming in next generation sequencing (NG sequencing). This sequence is commonly known as an adapter. The sequence of the adapter is specific to the system for NG sequencing. The skilled person will be able to determine the adapter sequence that is necessary for use in a particular NG sequencing system. Examples of adapters include Illumina adapters Read 2 and Read 2N, which are used with long cDNA chains and oligonucleotides, respectively, but any other pair of adapter sequence and NG sequencing system may be used. The sequence of the Read 2N adapter is CGGAGATGTGTATAAGAGACAG (SEQ ID NO: 6).

In another embodiment, the nucleic acid further comprises a sequence that is complementary to that of a template switching oligonucleotide. The sequence that is complementary to that of a template switching oligonucleotide, as used herein, is a capture sequence that anneals to a template switching oligonucleotide or switch oligo and so that the nucleic acid of the detection agent is extended by the reverse transcriptase (e.g. Superscript II) when used in the assays according to the invention. The term template switching oligonucleotide or switch oligo is defined in more detail in the context of the assays of the invention. An example of the sequence that is complementary to that of a template switching oligonucleotide is CCCATATAAGAAA (SEQ ID NO: 7). The three adenosines at the carboxy terminus may have a phosphorothioate bond for stabilisation.

In a particular embodiment, the nucleic acid which comprises a specific tag sequence comprises or consists of the sequence shown as SEQ ID NO: 8 (CGGAGATGTGTATAAGAGACAGACGTGACTACACGAATCAATCTGTGCTAGA CTGCCCCATATAAGAAA).

In a particular embodiment, the detection agent comprises an alpaca-anti-mouse IgG2a Fc monoclonal nanobody TP923 which is labelled with a nucleic acid having the sequence shown as SEQ ID NO: 8.

The present invention also contemplates that a detection agent may comprise an anti-IgG antibody labelled with an RNA sequence which comprises a specific ribonucleotide sequence and a poly(A) tail.

In another embodiment, the detection agent comprises a moiety that is suitable for binding specifically an antibody and which is labelled with a nucleic acid which comprises a specific tag sequence and further comprises a poly(A) tail, wherein the nucleic acid is RNA.

Examples of specific RNA tag sequences include, without limitation, the following sequences:

(SEQ ID NO: 3)
5′-UCACCCCUCAACAACUAGCAAAGGCAGCCCCAUAAACACACAGUAUGU
UUUUUGAAAAAAAAAAAAAAAAAAAAA-3′
(SEQ ID NO: 4)
5′-UAGGAAAGUUGGUCUUCGCCAUCAUGGCAGUUGCUUGCAAUGUAA
UUUUCAGUUAAAAAAAAAAAAAAAAAAAAAA-3′

The specific RNA tag sequence may encode the anti-IgG antibody. The anti-IgG antibody may be one of the nanobodies described previously.

It will be appreciated that the specific RNA tag sequence may be at least 450 nucleotides long.

The anti-IgG antibody and the specific RNA tag sequence may be linked together via a puromycin. The anti-IgG antibody and the RNA sequence may be linked together via an oligo linker containing psoralen and puromycin at its 5′ and 3′ end, respectively, followed by a stop codon. The RNA sequence may comprise a short sequence for hybridising and crosslinking with the oligo linker containing psoralen and puromycin at its 5′ and 3′ end, respectively, followed by a stop codon. The oligo linker may comprise 15 deoxyadenosines (dA15) located at the centre. This stretch of dA15 is suitable for purification purposes.

The detection agents described previously are suitable for use in the assays of the invention in combination with a bispecific reagent which has a second domain that comprises a target antigen. Where the bispecific reagent comprises a second domain which binds specifically to IgG, the present invention also contemplates a detection agent that comprises a target antigen which is labelled with a nucleic acid which comprises a specific tag sequence.

Thus, in another embodiment, the detection agent comprises a target antigen which is labelled with a nucleic acid which comprises a specific tag sequence.

The terms “target antigen”, “nucleic acid” and “specific tag sequence” have been defined have been defined previously in the context of the bispecific reagent of the invention and the detection agent of the invention, and the particular features and embodiments described therein are equally applicable to this embodiment. Where necessary, the skilled person will be able to make the changes necessary to adapt these elements for use in this embodiment.

5. Complexes C3 and D3

In another aspect, the present invention relates to a third complex, which comprises:
a) an antibody-producing cell;
b) a bispecific reagent according to the invention, bound to the antibody-producing cell via the antigen expressed by the antibody-producing cell;
c) an antibody which:
   (i) is secreted by the antibody-producing cell, and
   (ii) specifically binds the target antigen,
   wherein the antibody is bound to the bispecific reagent via the second domain thereof; and
d) a detection agent according to the invention, which is bound to the antibody of (c).

Where the second domain of the bispecific reagent comprises a target antigen, and the antibody secreted by the antibody-producing cell is bound specifically to the target antigen, then the detection agent the detection agent may comprise an anti-IgG antibody labelled with an RNA sequence which comprises a specific ribonucleotide sequence and a poly(A) tail.

Thus, the third complex, i.e. the complex C3, may comprise:
a) an antibody-producing cell;
b) a bispecific reagent according to the invention, which comprises a target antigen, bound to the antibody-producing cell via the antigen expressed by the antibody-producing cell;
c) an antibody which:
   (i) is secreted by the antibody-producing cell, and
   (ii) specifically binds the target antigen, wherein the antibody is bound to the bispecific reagent via the target antigen thereof; and
d) which comprises an anti-IgG antibody labelled with an RNA sequence which comprises a specific tag sequence and a poly(A) tail, which is bound to the antibody of (c).

Alternatively, where the second domain of the bispecific reagent comprises a binding domain which binds specifically to IgG, the antibody secreted by the antibody-producing cell is bound specifically to this.

Thus, the third complex, i.e. complex D3, may comprise:
a) an antibody-producing cell;
b) a bispecific reagent according to the invention, which comprises a binding domain which binds specifically to IgG, bound to the antibody-producing cell via the binding domain which binds specifically to IgG;
c) an antibody which:
   (i) is secreted by the antibody-producing cell, and
   (ii) specifically binds the target antigen,
   wherein the antibody is bound to the bispecific reagent via the binding domain which binds specifically to IgG; and
d) a detection agent which comprises the target antigen labelled with an RNA sequence which comprises a specific tag sequence and a poly(A) tail, which is bound to the antibody of (c).

It will be immediately appreciated that three of the elements of the complexes C3 and D3, i.e. the antibody-producing cell (a), the bispecific reagent according to the invention (b), which is bound to the antibody-producing cell via the antigen expressed by the antibody-producing cell, and the antibody (c), which (i) is secreted by the antibody-producing cell, and (ii) specifically binds the target antigen, wherein the antibody is bound to the bispecific reagent via the second domain thereof, constitute the complex C2 or D2 of the invention. Accordingly, the definitions, particular features and embodiments that have been described in the context of the complexes C2 or D2 of the invention are equally applicable to the complexes C3 and D3 of the invention.

The complexes C3 and D3 additionally comprises a third element (d), i.e. a detection agent according to the invention, which is bound to the antibody of (c).

The definitions, particular features and embodiments of the detection agent have been described in the context of the detection agent of the invention are equally applicable to the complexes C3 and D3 of the invention.

6. Assay

The reagents and complexes described herein may be advantageously used in a method for interrogating a population of antibody-producing cells to identify those cells which secrete antibodies which are specific to a target antigen of interest.

In another aspect, the present invention relates to an assay for identifying an antibody-producing cell which produces an antibody that binds specifically to a target antigen, hereinafter “the assay of the invention”, which comprises the following steps:
(i) providing a population of antibody-producing cells;
(ii) binding a bispecific reagent according to the invention to the cells;
(iii) incubating the cells from step (ii) with a target antigen;
(iv) adding a detection agent according to the invention to the cells from step (iii);
(v) partitioning the cells from (iv) into partitions, wherein each partition contains a single cell and a unique barcode molecule;
(vi) performing reverse transcription such that all RNA molecules in the cell within the partition and the RNA sequence of the detection reagent (if present) are barcoded with the unique barcode molecule;
(vii) disrupting the partitions and pooling the barcoded nucleic acid sequences from (vi)
(viii) analysing the pooled sequences to find sets of sequences with the same unique barcode which comprise:
   (a) a sequence encoding a heavy chain variable domain (VH);
   (b) a sequence encoding a light chain variable domain (VL); and
   (c) a sequence corresponding to the reverse transcript of the RNA sequence of the detection agent

The terms “antibody-producing cell”, “bispecific reagent”, “target antigen”, and “detection reagent have been described in detail previously in the context of other aspects of the invention and their definitions, particular features and embodiments apply equally to the assay of the invention.

In a first step, the assay of the invention comprises providing a population of antibody-producing cells. The population containing antibody-producing cells may be from a biological fluid. The biological fluid may comprise blood or lymph fluid. The population containing antibody-producing cells may be a peripheral blood mononuclear cell (PBMC) sample.

The population of antibody-producing cells may be produced by immunising an animal, e.g. a mouse, with a target antigen. Several methods are available in the art for the immunisation of animals. In a particular embodiment, the animal is immunised using an expression plasmid encoding the target antigen following the genetic immunisation protocols developed by Aldevron (Aldevron Fargo, Fargo, N. Dak., USA).

The population of antibody-producing cells need not be pure. Thus the population of antibody-producing cells may contain at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 95%, at least 96%, at least 97%, at least 98%, at least 99%, or 100% of antibody-producing cells.

It may be particularly advantageous to use a population that has been enriched in antibody-producing cells. Thus, the assay of the invention may comprise a step of enriching a population containing antibody-producing cells in antibody-producing cells prior to step (i). This may be performed by various methods that are conventional in the art, such as FACS. Antigens expressed on the surface of antibody-producing cells may be used, including one or more of CD138, CD38, CD98, Sca-1, Ly6c1/2, Ly6k, CD28, transmembrane activator and calcium modulator and cyclophilin ligand interactor (TACI), B-cell maturation antigen (BCMA), and/or SLAM7. Other markers of proliferation may be used instead or in addition to the ASC markers to enrich samples for acutely proliferated plasmablasts.

In a second step, the assay of the invention comprises binding a bispecific reagent according to the invention to the antibody-producing cells. This may be attained by applying the bispecific reagent to the cells and, optionally, shaking gently to facilitate the bispecific reagent coming into contact with the antibody-producing cells. The unbound bispecific reagent may be conveniently washed-off. As a result, the antibody-producing cells and the bispecific reagent form a complex according to complex C1 or D1 of the invention.

In a third step, the assay of the invention comprises incubating the cells from the previous step with a target antigen. This may be attained by applying the target antigen to the cells and, optionally, shaking gently to facilitate the target antigen coming into contact with the antibody-producing cells. The unbound target antigen may be conveniently washed-off.

This step will result in the activation of the antibody-producing cells that are specific for the target antigen and the subsequent secretion of antibodies specific for the target antigen. The antibodies will be captured onto the same cell that produced and secreted it by means of the free domain of the bispecific reagent before they diffuse away from the cell. Since the antibody is secreted by the antibody-producing cell and specifically binds the target antigen, it will be readily appreciated that the complex formed after the third step is the complex C2 or D2 according to the invention.

The antibody-producing cells may be in a liquid or semi-solid medium. Any liquid media suitable for the culture of B cells may be used including, without limitation, B-Cell Medium (45% IMDM, 45% DMEM high glucose, 10% fetal bovine serum, 50 μM β-mercaptoethanol, 2 mM L-glutamine, 50 U/mL penicillin, 50 μg/mL streptomycin), Human Blood Cell Medium (Cell Applications, Inc.), R5 medium (RPMI 1640 with 5% human serum, 55 μM 2-mercaptoethanol, 2 mM L-glutamine, 100 U/ml penicillin, 100 μg/ml streptomycin, 10 mM HEPES, 1 mM sodium pyruvate and 1% MEM nonessential amino acids), and a medium containing RPMI 1640, 5% fetal calf serum, 2 mM L-glutamine, 1% penicillin/streptomycin, and 50 μM β-mercaptoethanol. Non-limiting examples of semi-solid media include MethoCult™ M3231 (Stemcell Technologies), AbeoClone™ CHO TCSC Semi-solid Medium (VWR) and CloneMedia (Molecular Devices), and a medium made by 1:1 mixing 2× Plasma Cell Isolation medium (Miltenyi Biotec) with AbeoClone™ Base Semi-solid Medium (2×) (VWR).

The unbound secreted antibodies may be washed-off.

After the third step, the population of antibody-producing cells will contain cells having the antibody that they secrete captured onto their surface as well as cells with no antibody bound onto their surface.

In a fourth step, the assay of the invention comprises adding a detection agent according to the invention to the cells from step (iii). This may be attained by applying the detection agent to the cells and, optionally, shaking gently to facilitate the detection agent coming into contact with the antibody-producing cells. The detection agent will bind to the antibody which specifically binds the target antigen and which is captured onto the surface of the antibody-producing cell by means of the bispecific reagent. The complex formed after the third step is the complex C3 or D3 according to the invention.

As will be appreciated, by incubating the cells from step (iii) simultaneously with different detection agents, it is possible to screen for cells secreting antibodies having different binding characteristics. For example, in the particular embodiment where the detection agent comprises the target antigen, the cells may be co-incubated with (i) a detection agent comprising the same target antigen (e.g. human) used to immunise the animal from which the cells originate, and (ii) a detection agent comprising the target antigen from a different species (e.g. mouse) to the target antigen. By having the two different detection agents conveniently labelled with a different specific RNA tag sequence, it is possible to screen for antibodies having complex specificities that are able to cross-react.

In another example, the detection agent comprises the target antigen and may be labelled with different specific RNA tag sequences. By adding the differently labelled detection agents at different time-points to the cells, on- and off-rates can be determined from the sequencing data.

These are no more than mere examples of the flexibility and potential of the assay of the invention. The person skilled in the art will readily know how to make variations to the assay in order to screen for antibodies having specific binding characteristics.

The unbound detection agent may be conveniently washed-off.

After the fourth step, the population of antibody-producing cells will contain cells having the antibody that they secrete captured onto their surface, which will have the detection agent bound to it, as well as cells with no antibody bound onto their surface and thus with no detection agent bound to them. It will be appreciated that the detection agent will be bound only to those antibody-producing cells onto which surface the antibody which they secrete is captured by means of the bispecific reagent. This is particularly advantageous because it enables the detection of the individual cell which produces the antibody with the required specificity. Importantly, this enables the retrieval of the genetic information encoding the antibody.

The population of antibody-producing cells may contain at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 95%, at least 96%, at least 97%, at least 98%, at least 99%, or 100% of antibody-producing cells comprising the detection agent bound to them.

In a fifth step, the assay of the invention comprises partitioning the cells obtained from the fourth step into partitions, wherein each partition contains a single cell and a unique barcode molecule.

As will be appreciated, as a result of partitioning the cells each partition contains a single antibody-producing cell and a unique barcode molecule but the detection reagent may not be present. The detection reagent is only present in those partitions that contain a single antibody-secreting cell which produces an antibody which specifically binds the target antigen.

The term “partition”, as used herein, refers to discrete compartments or partitions, which are used indistinctly herein. Each partition maintains a separation of its own contents from the contents of other partitions. A partition may be a droplet, microvesicle, or a vessel. When the partitions refer to a droplet, they may comprise an aqueous fluid within a non-aqueous continuous phase, for example, an oil phase. When the partitions refer to a microvesicle, it has an outer barrier surrounding an inner fluid centre or core, or, in some cases, they may comprise a porous matrix that is capable of entraining and/or retaining materials within its matrix. When the partitions refer to a container or vessel, these may be wells, microwells, tubes, vials, through ports in nanoarray substrates, for example, BioTrove nanoarrays, or other containers.

The partitions described herein may comprise small volumes, such as less than 10 μL, less than 5 μL, less than 1 μL, less than 500 nL, less than 100 nL, less than 50 nL, less than 10 nL, less than 5 nL, less than 1 nL, less than 900 picoliters (pL), less than 800 pL, less than 700 pL, less than 600 pL, less than 500 pL, less than 400 pL, less than 300 pL, less than 200 pL, less than 100 pL, less than 50 pL, less than 20 pL, less than 10 pL, less than 1 pL, or even less. Alternatively or in combination, the partitions may be of uniform size or heterogeneous size, with a diameter less than 1 mm, less than 500 μm, less than 250 μm, less than 100 μm, less than 90 μm, less than 80 μm, less than 70 μm, less than 60 μm, less than 50 μm, less than 40 μm, less than 30 μm, less than 20 μm, less than 10 μm, or less than 5 μm, or at least about 1 μm.

The term “partitioning”, as used herein, refers to the compartmentalisation, depositing or partitioning individual cells into distinct compartments or partitions. Any method for partitioning a population cells into individual cells, i.e. by controlling the occupancy of the resulting partitions (i.e. number of cells per partition), is suitable for the purposes of the present invention. These include, without limitation, the use of techniques based on microfluidic networks, droplets, microwell plates, and automatic collection of cells using capillaries, magnets, an electric field, or a punching probe. Partitioning of cells can be conveniently carried out using commercially available instruments, such as the ddSEQ Single-Cell Isolator, by Bio-Rad (Hercules, Calif., USA) and Illumina, (San Diego, Calif., USA), the Chromium system, by 10× Genomics (Pleasanton, Calif., USA), the Rhapsody Single-Cell Analysis System, by Becton, Dickinson and Company (BD, Franklin Lakes, N.J., USA), the Tapestri Platform (MissionBio, San Francisco, Calif., USA).

In order to ensure that those partitions that are occupied are primarily occupied by a single cell, it may be desirable that partitions contain less than one cell per partition. Thus, the majority of occupied partitions may include no more than one cell per occupied partition. In some cases, the partitioning process is conducted such that fewer than 25%, fewer than 20%, fewer than 15%, fewer than 10%, fewer than 5%, or fewer than 1% of the occupied partitions contain more than one cell.

Each partition contains a single cell and a unique barcode molecule. The term “barcode” or “barcode molecule”, as used herein, refers to a sequence, a label, or identifier that can be part of an analyte to convey information about the analyte. Barcodes can allow for identification and/or quantification of individual sequencing-reads in real time. The barcode is unique in the sense that all the barcodes in one partition are the same, but the barcodes in each partition are different from each other. Thus, in operation, the same barcode will be incorporated to all the cDNA products that are obtained by RT-PCR in a single cell. A barcode can be a sequence tag attached to an analyte (e.g. nucleic acid molecule) or a combination of the tag in addition to an endogenous characteristic of the analyte (e.g. size of the analyte or end sequence). Barcodes can have a variety of different formats, for example, barcodes can include: polynucleotide barcodes; random nucleic acid and/or amino acid sequences; and synthetic nucleic acid and/or amino acid sequences. A barcode can be attached to an analyte in a reversible or irreversible manner. The barcode can be added to, for example, a fragment of a deoxyribonucleic acid (DNA) or ribonucleic acid (RNA) sample before, during, and/or after sequencing of the sample. The barcode may be generated in a combinatorial manner. Barcodes that may be used with methods of the present disclosure are described in, for example, US Patent Pub. No. 2014/0378350.

The barcode molecule may be a polynucleotide. The length of a polynucleotidic barcode molecule may be at least 5, at least 6, at least 7, at least 8, at least 9, at least 10, at least 11, at least 12, at least 13, at least 14, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 160, at least 170, at least 180, at least 190, at least 200, at least 210, at least 220, at least 230, at least 240, at least 250 nucleotides, at least 500 nucleotides, or longer.

The structure of the barcode oligonucleotides may include a number of sequence elements useful in the processing of the nucleic acids from the co-partitioned cells in addition to the oligonucleotide barcode sequence. These sequences include targeted or random/universal amplification primer sequences for amplifying the genomic DNA from the individual cells within the partitions while attaching the associated barcode sequences, sequencing primers or primer recognition sites, hybridisation or probing sequences, e.g. for identification of presence of the sequences or for pulling down barcoded nucleic acids, or any of a number of other potential functional sequences.

One example of a barcode oligonucleotide for use in RNA analysis is coupled to a bead by a releasable linkage, such as a disulfide linker. The oligonucleotide may include functional sequences that are used in subsequent processing. As will be appreciated, the functional sequences may be selected to be compatible with a variety of different sequencing systems, such as 454 Sequencing, Ion Torrent Proton or PGM, Illumina X10, etc., and the requirements thereof. A barcode sequence is included within the structure for use in barcoding the sample RNA. An mRNA specific priming sequence, such as poly-T sequence may also be included in the oligonucleotide structure. Other sequences may be used as primer sequences in the context of the present invention, including, without limitation, a sequence which is complementary to a sequence encoding one of the IgG constant domains, a sequence which is complementary to the specific RNA tag sequence comprised in the detection agent, a sequence which is complementary to a sequence encoding one of the IgG variable domains, and combinations thereof.

An anchoring sequence segment may be included to ensure that the poly-T sequence hybridises at the sequence end of the mRNA. This anchoring sequence can include a random short sequence of nucleotides, e.g., 1-mer, 2-mer, 3-mer or longer sequence, which will ensure that the poly-T segment is more likely to hybridise at the sequence end of the poly-A tail of the mRNA.

An additional sequence segment may be provided within the oligonucleotide sequence. In some cases, this additional sequence may provide a unique molecular identifier (UMI) sequence segment, such as a random sequence (for example, a random N-mer sequence) that varies across individual oligonucleotides coupled to a single partition, whereas the barcode sequence may be constant among oligonucleotides tethered to an individual partition. This UMI serves to provide a unique identifier of the starting mRNA molecule that was captured, in order to allow quantitation of the number of original expressed RNA. As will be appreciated, individual partitions may include tens to hundreds of thousands or even millions of individual oligonucleotide molecules, where the barcode molecule may be constant or relatively constant for a given partition, but where the UMI will vary across an individual partition. This UMI sequence segment may include from 5 to about 8 or more nucleotides within the sequence of the oligonucleotides. In some cases, the UMI sequence segment may be 2, 3, 4, 5, 6, 7, 8, 9, 10, 11, 12, 13, 14, 15, 16, 17, 18, 19 or 20 nucleotides in length or longer.

In some cases, it may be desirable to incorporate multiple different barcodes within a given partition, either attached to a single or to multiple beads within the partition. For example, in some cases, a mixed, but known barcode sequences set may provide greater assurance of identification in the subsequent processing, e.g., by providing a stronger address or attribution of the barcodes to a given partition, as a duplicate or independent confirmation of the output from a given partition.

The oligonucleotides may be releasable from the beads upon the application of a particular stimulus to the beads. In some cases, the stimulus may be a photo-stimulus, e.g. through cleavage of a photo-labile linkage that releases the barcode molecule. In other cases, a thermal stimulus may be used, where elevation of the temperature of the beads environment will result in cleavage of a linkage or other release of the barcode molecule from the beads. In still other cases, a chemical stimulus is used that cleaves a linkage of the oligonucleotides to the beads, or otherwise results in release of the barcode molecule from the beads, such as through exposure to a reducing agent, e.g. DTT.

The barcode is delivered to a partition via a bead. The term “bead”, as used herein, refers to a microparticle having a diameter of between 1 μm and 1 mm, irrespective of the precise interior or exterior structure. Non-limiting examples of beads include a microcapsule and a microsphere. The bead may be porous, non-porous, solid, semi-solid, semi-fluidic, or fluidic. The bead may be dissolvable, disruptable, or degradable. The bead may not be degradable. The bead may be a gel bead. The gel bead may be a hydrogel bead. The gel bead may be formed from molecular precursors, such as a polymeric or monomeric species. A semi-solid bead may be a liposomal bead. A solid bead may comprise metals including iron oxide, gold, and silver. The bead may be a silica bead. The bead may be rigid. In some cases, the bead may be flexible and/or compressible.

Beads may be of uniform size or heterogeneous size. In some cases, the diameter of a bead may be less than 1 mm, less than 500 μm, less than 250 μm, less than 100 μm, less than 90 μm, less than 80 μm, less than 70 μm, less than 60 μm, less than 50 μm, less than 40 μm, less than 30 μm, less than 20 μm, less than 10 μm, or less than 5 μm, or at least about 1 μm.

Beads may be of uniform size or heterogeneous size. In some cases, the diameter of a bead may be at least about 1 μm, 5 μm, 10 μm, 20 μm, 30 μm, 40 μm, 50 μm, 60 μm, 70 μm, 80 μm, 90 μm, 100 μm, 250 μm, 500 μm, or 1 mm.

Any suitable number of barcode molecules can be associated with a bead such that the barcoded molecules are present in the partition at a predefined concentration. Such predefined concentration may be selected to facilitate certain reactions for generating a sequencing library, such as amplification, within the partition. The population of beads may provide a diverse barcode sequence library that includes at least 1,000 different barcode sequences, at least 5,000 different barcode sequences, at least 10,000 different barcode sequences, at least at least 50,000 different barcode sequences, at least 100,000 different barcode sequences, at least 1,000,000 different barcode sequences, at least 5,000,000 different barcode sequences, or at least 10,000,000 different barcode sequences.

Methods for connecting a barcode molecule to a bead are known in the art.

As will be appreciated, the above-described occupancy rates are also applicable to partitions that include both cells and additional reagents, including, without limitation, microcapsules or beads carrying barcoded oligonucleotides. At least 5%, at least 10%, at least 20%, at least 30%, at least 40%, at least 50%, at least 60%, at least 70%, at least 80%, at least 85%, at least 90%, at least 95%, at least 96%, at least 97%, at least 98%, at least 99%, or at least 100% of the partitions contain both a microcapsule comprising barcode molecules and a cell.

In addition to beads, other reagents may also be co-partitioned with the cells. The cells may be partitioned along with lysis reagents in order to release the contents of the cells within the partition. In such cases, the lysis agents can be contacted with the cell suspension concurrently with, or immediately prior to the introduction of the cells into the partitioning junction/droplet generation zone. Examples of lysis agents include bioactive reagents, such as lysis enzymes, for example, lysozymes, achromopeptidase, lysostaphin, labiase, kitalase, lyticase, and a variety of other commercially available lysis enzymes. Other lysis agents may additionally or alternatively be co-partitioned with the cells to cause the release of the cell's contents into the partitions. For example, in some cases, surfactant based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g. non-emulsion based partitioning such as encapsulation of cells that may be in addition to or in place of droplet partitioning, where any pore size of the encapsulate is sufficiently small to retain nucleic acid fragments of a desired size, following cellular disruption.

In addition to the lysis agents co-partitioned with the cells described above, other reagents can also be co-partitioned with the cells, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated cells, the cells may be exposed to an appropriate stimulus to release the cells or their contents from a co-partitioned microcapsule. For example, in some cases, a chemical stimulus may be co-partitioned along with an encapsulated cell to allow for the degradation of the microcapsule and release of the cell or its contents into the larger partition. This stimulus may be the same as the stimulus described elsewhere herein for release of oligonucleotides from their respective bead (e.g. microcapsule). Alternatively, this may be a different and non-overlapping stimulus, in order to allow an encapsulated cell to be released into a partition at a different time from the release of barcode molecule into the same partition.

In some cases, it may be desirable to keep the barcode molecule attached to the bead (e.g. microcapsule). For example, the partition-bound oligonucleotides may be used to hybridise and capture the mRNA on the solid phase of the partition in order to facilitate the separation of the RNA from other cell contents.

Additional reagents may also be co-partitioned with the cells, such as endonucleases to fragment the cell's DNA, DNA polymerase enzymes and dNTPs used to amplify the cell's nucleic acid fragments and to attach the barcode oligonucleotides to the amplified fragments. Additional reagents may also include reverse transcriptase enzymes, including enzymes with terminal transferase activity, primers and oligonucleotides, and switch oligonucleotides, also referred to herein as “switch oligos” or “template switching oligonucleotides”, which can be used for template switching. Switching can be used to increase the length of a cDNA. Template switching can be used to append a predefined nucleic acid sequence to the cDNA. In one example of template switching, cDNA can be generated from reverse transcription of a template, e.g. cellular mRNA, where a reverse transcriptase with terminal transferase activity can add additional nucleotides, e.g., polyC, to the cDNA in a template independent manner.

The additional reagents may be delivered to a partition by means of additional beads, or together with the barcode molecules.

Once the co-partitioned cells are lysed and their contents released into their respective partitions, the nucleic acids contained therein may be further processed within the partitions. The barcode molecules disposed on the bead may be used to barcode and amplify fragments of those nucleic acids.

Thus, in a sixth step, the assay of the invention comprises performing reverse transcription such that all mRNA molecules in the cell within the partition and the RNA sequence of the detection reagent (if present) are barcoded with the unique barcode molecule.

The reverse transcriptase may be conveniently provided within the partition. The reverse transcription reaction may be performed using any commercially available reverse transcriptase according to conventional methods, which include a step of annealing and elongation.

The reverse transcription may be performed using an oligonucleotide that forms part of the barcode molecule as priming agent.

The primer portion of the barcode molecule can anneal to a complementary region of a cell's nucleic acid. Extension reaction reagents, e.g., DNA polymerase, nucleoside triphosphates, co-factors (e.g., Mg²⁺ or Mn²⁺), that are also co-partitioned with the cells and beads, then extend the primer sequence using the cell's nucleic acid as a template, to produce a complementary fragment to the strand of the cell's nucleic acid to which the primer annealed, which complementary fragment includes the oligonucleotide and its associated barcode sequence. Annealing and extension of multiple primers to different portions of the cell's nucleic acids will result in a large pool of overlapping complementary fragments of the nucleic acid, each possessing its own barcode sequence indicative of the partition in which it was created. In some cases, these complementary fragments may themselves be used as a template primed by the oligonucleotides present in the partition to produce a complement of the complement that again, includes the barcode sequence. In some cases, this replication process is configured such that when the first complement is duplicated, it produces two complementary sequences at or near its termini, to allow formation of a hairpin structure or partial hairpin structure, the reduces the ability of the molecule to be the basis for producing further iterative copies.

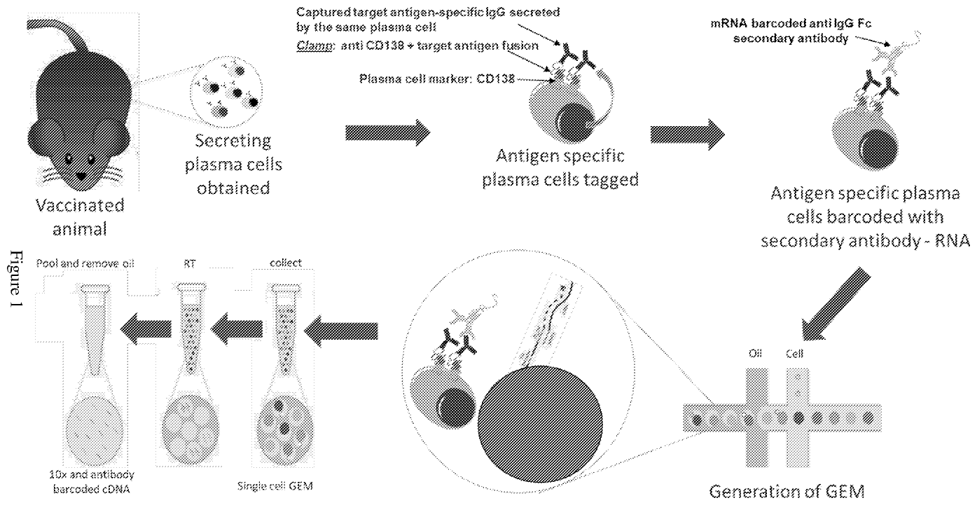

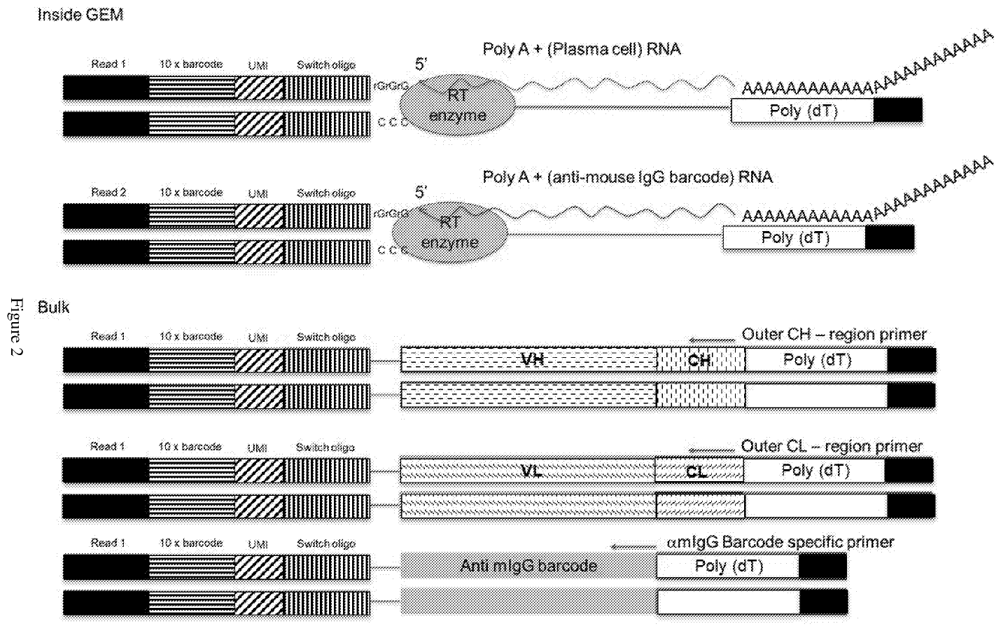

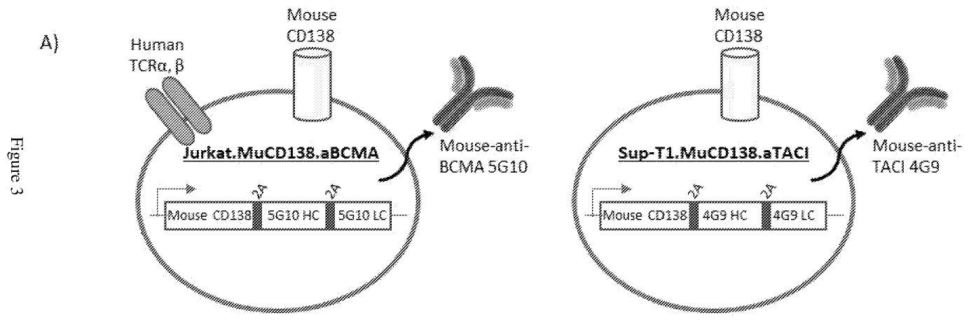

In operation, and with reference to FIGS. 1 and 2, an antibody-producing cell is co-partitioned along with a barcode bearing bead and lysed while the barcoded oligonucleotides are released from the bead. The poly-T portion of the released barcode oligonucleotide then hybridises to the poly-A tail of each mRNA molecule present in the cell, and to the poly-A tail of the specific tag sequence comprised in the detection agent, if present in the partition. The poly-T segment then primes the reverse transcription of the mRNA to produce a cDNA transcript of the mRNA, but which includes each of the sequence segments of the barcode oligonucleotide. Again, because the oligonucleotide includes an anchoring sequence, it will more likely hybridise to and prime reverse transcription at the sequence end of the poly-A tail of the mRNA. Within any given partition, all of the cDNA transcripts of the individual mRNA molecules will include a common or unique barcode sequence segment. However, by including the unique random N-mer sequence, the transcripts made from different mRNA molecules within a given partition will vary at this unique sequence. This provides a quantitation feature that can be identifiable even following any subsequent amplification of the contents of a given partition, e.g., the number of unique segments associated with a common barcode can be indicative of the quantity of mRNA originating from a single partition, and thus, a single cell. As noted above, the transcripts are then amplified, cleaned up and sequenced to identify the sequence of the cDNA transcript of the mRNA, as well as to sequence the barcode segment and the unique sequence segment.

While a poly-T primer sequence is described, other targeted or random priming sequences may also be used in priming the reverse transcription reaction. In some cases, the primer sequence can be a gene specific primer sequence which targets specific genes for reverse transcription. Such target genes may comprise immunoglobulin genes.

Optionally, a step of amplification by polymerase chain reaction (PCR) may be performed prior to the disruption of the partitions and pooling of the barcoded nucleic acids with the purpose of enriching a subset of nucleic acids corresponding to specific sequences encoding (i) an IgG heavy chain region, (ii) an IgG light chain region, and (iii) a sequence corresponding to the reverse transcript of the RNA specific tag sequence of the detection agent. One or more gene specific primers can be used together with the barcode molecule for primer extension using the cDNA molecule as a template. The sequences of these primers will be readily determined by the person skilled in the art. The primers to amplify the IgG heavy chain region may comprise an oligonucleotide having a sequence complementary to a sequence encoding IgG heavy chain constant region (CH), or having a sequence specific for the 3′end of the IgG heavy chain variable region (VH), and an oligonucleotide having a sequence specific for the barcode molecule. The primers to amplify the IgG light chain region may comprise an oligonucleotide having a sequence complementary to a sequence encoding IgG light chain constant region (CL), or having a sequence complementary to a sequence encoding IgG light chain variable region (VL), and an oligonucleotide having a sequence specific for the barcode molecule. The primers to amplify the specific RNA tag sequence of the detection agent may comprise an oligonucleotide having a sequence complementary to the RNA specific tag sequence, and an oligonucleotide having a sequence specific for the barcode molecule. The primers may conveniently be provided or delivered to the partition with a bead (e.g. microcapsule).

In the event where the specific RNA tag sequence or the detection agent encodes an anti-IgG antibody, the primer to amplify this sequence is a sequence complementary to said anti-IgG antibody. The particular embodiments of the anti-IgG antibody have been described in the context of the detection agent of the invention and apply equally to the assay of the invention

The amplification may be carried out for at least 5, at least 10, at least 15, at least 20, at least 25, at least 30, at least 40 or more cycles. In general, the amplification of the cell's nucleic acids is carried out until the barcoded overlapping fragments within the partition constitute at least 1× coverage of the particular portion or all of the cell's genome, at least 2×, at least 3×, at least 4×, at least 5×, at least 10×, at least 20×, at least 40× or more coverage of the genome or its relevant portion of interest.

Any of a variety of polymerases can be used in embodiments herein for primer extension, including, without limitation, exonuclease minus DNA Polymerase I large (Klenow)

Fragment, Phi29 DNA polymerase, Taq DNA Polymerase, T4 DNA polymerase, T7 DNA polymerase, and the like. Further examples of polymerase enzymes that can be used in embodiments herein include thermostable polymerases. In some embodiments, a hot start polymerase is used. A hot start polymerase is a modified form of a DNA polymerase that can be activated by incubation at elevated temperatures.

As previously noted, each distinct labelling sequence may correspond to a single antibody-secreting cell. Enrichment increases accuracy and sensitivity of methods for sequencing immunoglobulin genes at a single cell level. Enrichment may lead to greater than or equal to 25%, 30%, 35%, 40%, 45%, 50%, 55%, 60%, 65%, 70%, 75%, 80%, 85%, 90%, 95% or more of total sequencing reads mapping to an immunoglobulin gene.

The reverse transcription may be carried out by 5′ Rapid Amplification of cDNA Ends (5′-RACE). 5′-RACE, or “one-sided” PCR or “anchored” PCR, is a technique that facilitates the isolation and characterisation of 5′ ends from low-copy transcripts.

Following the generation of barcoded template polynucleotides or derivatives (e.g. amplification products) thereof, subsequent operations may be performed, including purification (e.g. via solid phase reversible immobilization (SPRI)) or further processing (e.g. shearing, addition of functional sequences, and subsequent amplification, e.g. by PCR). These operations may occur in bulk, for example, outside the partition.

Thus, in a seventh step, the assay of the invention comprises disrupting the partitions and pooling the barcoded nucleic acid sequences from the sixth step.

The partitions may be disrupted by any suitable means, such as by mechanical disruption, by an increase in pressure or by chemical disruption.

As will be understood, as a result of pooling the barcoded nucleic acid sequences from the sixth step, there is obtained a mixture of all of the cDNA transcripts of the individual mRNA molecules originally contained in the antibody-producing cells of the population of the first step. Thus there will be a mixture of unique barcode sequence segments, each identifying a different cell of origin.

Optionally, a step of amplification by polymerase chain reaction (PCR) may be performed in bulk after pooling the barcoded nucleic acids in order to amplify nucleic acids corresponding to specific sequences encoding (i) an IgG heavy chain region, (ii) an IgG light chain region, and (iii) a sequence corresponding to the reverse transcript of the RNA specific tag sequence of the detection agent. One or more gene specific primers can be used together with the barcode molecule for primer extension using the cDNA molecule as a template. The sequences of these primers will be readily determined by the person skilled in the art. The primers to amplify the IgG heavy chain region may comprise an oligonucleotide having a sequence complementary to a sequence encoding IgG heavy chain constant region (CH), or having a sequence specific for the 3′end of the IgG heavy chain variable region (VH), and an oligonucleotide having a sequence specific for the barcode molecule. The primers to amplify the specific RNA tag sequence of the detection agent may comprise an oligonucleotide having a sequence complementary to the RNA specific tag sequence, and an oligonucleotide having a sequence specific for the barcode molecule. The primers may conveniently be provided or delivered to the partition with a bead (e.g. microcapsule).

In the event where the specific RNA tag sequence or the detection agent encodes an anti-IgG antibody, the primer to amplify this sequence is a sequence complementary to said anti-IgG antibody. The particular embodiments of the anti-IgG antibody have been described in the context of the detection agent of the invention and apply equally to the assay of the invention

The amplification may be carried out for at least 5, at least 10, at least 15, at least 20, at least 25, at least 30, at least 40 or more cycles. In general, the amplification of the cell's nucleic acids is carried out until the barcoded overlapping fragments within the partition constitute at least 1× coverage of the particular portion or all of the cell's genome, at least 2×, at least 3×, at least 4×, at least 5×, at least 10×, at least 20×, at least 40× or more coverage of the genome or its relevant portion of interest.

Any of a variety of polymerases can be used in embodiments herein for primer extension, including, without limitation, exonuclease minus DNA Polymerase I large (Klenow)

Fragment, Phi29 DNA polymerase, Taq DNA Polymerase, T4 DNA polymerase, T7 DNA polymerase, and the like. Further examples of polymerase enzymes that can be used in embodiments herein include thermostable polymerases. In some embodiments, a hot start polymerase is used. A hot start polymerase is a modified form of a DNA polymerase that can be activated by incubation at elevated temperatures.

As previously noted, each distinct sequence may correspond to a single antibody-producing cell. Enrichment increases accuracy and sensitivity of methods for sequencing immunoglobulin genes at a single cell level. Enrichment may lead to greater than or equal to 25%, 30%, 35%, 40%, 45%, 50%, 55%, 60%, 65%, 70%, 75%, 80%, 85%, 90%, 95% or more of total sequencing reads mapping to an immunoglobulin gene.

In an eighth step, the assay according to the invention comprises analysing the pooled sequences to find sets of sequences with the same unique barcode which comprise:
(a) a sequence encoding an IgG heavy chain;
(b) a sequence encoding an IgG light chain; and
(c) a sequence corresponding to the reverse transcript of the RNA sequence of the detection agent.

In a particular embodiment, the sets of sequences with the same unique barcode comprise:
(a) a sequence encoding a heavy chain variable domain (VH);
(b) a sequence encoding a light chain variable domain (VL); and
(c) a sequence corresponding to the reverse transcript of the RNA sequence of the detection agent

The analysis of the eighth step may comprise a step of DNA sequencing. The term “sequencing”, as used herein, refers to methods and technologies for determining the sequence of nucleotide bases in one or more polynucleotides. The polynucleotides can be, for example, deoxyribonucleic acid (DNA) or variants or derivatives thereof, such as single stranded DNA. DNA sequencing can be performed by any technique and system currently available, such as, without limitation, next generation sequencing or high throughput sequencing techniques, including Roche 454 pyrosequencing and other sequencing technologies by Illumina, Pacific Biosciences, Oxford Nanopore, and Life Technologies.

The DNA sequences obtained from this step all contain a barcode and a UMI.

By assembling the sequences according to their barcode, sequences can be grouped according to their starting antibody-secreting cell. In any given group of DNA sequences, the presence of the specific RNA tag sequence of the detection agent indicates that the antibody-specific sequences contained in the same group encode an antibody which binds specifically to the target antigen.

Where detection agents comprising different specific RNA tag sequences were incubated with the antibody-producing cells in step (iii), the presence and abundance of the different specific RNA sequences can be evaluated in order to obtain information about the properties of the antibody, including the antibody's complex specificities and binding kinetics.

The analysis may also include further assembling sequences according to each UMI in order group sequences according to their starting mRNA molecule, then merging highly similar assembled sequences. This step allows quantitation of the number of original expressed RNA transcripts, i.e. quantitation of gene expression levels.

7. Kit

The present invention also provides a kit for use in the assay of the invention, hereinafter “the kit of the invention”, which comprises the bispecific reagent of the invention and the detection agent of the invention.

The kit may comprise one or more components selected from the group consisting of partitioning fluids, barcode molecule libraries, which may be associated or not with beads (e.g. microcapsules), reagents for disrupting cells, reagents for amplifying nucleic acids, and any other component required to carry out the assay of the invention.

Instructions for using the kit of the invention according to the assay of the invention may also be provided.

The invention will now be further described by way of Examples, which are meant to serve to assist one of ordinary skill in the art in carrying out the invention and are not intended in any way to limit the scope of the invention.

### EXAMPLES

### Example 1: Screening of CD21-Specific Antibodies by Cell Sorting

In order to generate antibodies specific to CD21, a group of three mice is immunised using an expression plasmid encoding CD21 using the genetic immunisation protocols developed by Aldevron (Aldevron Fargo, Fargo, N. Dak., USA).

The serum from individual immunised mice is evaluated for binding to purified recombinant CD21 and to target CD21 expressed on cell surface via ELISA and flow cytometry, respectively. The spleen and bone marrow are harvested from those mice which show the highest sera conversion against CD21.

The spleen and bone marrow are disrupted into a single cell suspension. Those cells with surface IgG expression, i.e. memory B cells, are depleted from the cell suspension using an excess of Dynabeads® goat-anti mouse IgG (ThermoFisher). All cells that are not bound on the beads are collected via magnetic separation where all beads and bound cells are retained on a magnet. This population of unbound cells is enriched in plasma cells.

A conjugate between rat-anti mouse CD138 monoclonal antibody (clone 281-2, BD Biosciences) and the purified recombinant CD21 is prepared using the Protein-Protein Conjugation Kit from Tri-Link Biotechnologies. The conjugated rat-anti mouse CD138-CD21 is mixed thoroughly into semi-solid methylcellulose-based medium (MethoCult™ M3231 from Stemcell Technologies).

The cells that did not bind to the beads and were collected by magnetic separation are mixed into the modified semi-solid medium and incubated at 37° C., 5% CO₂ for between 1 and 4 hours without disturbance. During this process, the conjugated Rat-anti Mouse CD138-CD21 binds to CD138 on the surface of plasma cells. The conjugate bound to CD138 has CD21 moiety free so that it can act as an antibody trap. Specifically, if a plasma cell secrets IgG specific to CD21, such secreted IgG, especially in the proximity of the corresponding cell, can be captured on the surface of the same cell.

Following the incubation of the plasma cells in the modified semi-solid medium, the cells are diluted with Hanks' Balanced Salt Solution (HBSS, no calcium and magnesium), and mix gently with Fluorescein (FITC)-labelled AffiniPure Goat-anti-Mouse IgG (subclasses IgG1, IgG2a, IgG2b, and IgG3), Fey Fragment Specific (Jackson ImmunoResearch) and Phycoerythrin (PE)-labelled Goat-anti-Mouse CD184 (Bio-Rad). CD184 is highly expressed on the surface of Mouse plasma cells. Subsequently, the stained cells are washed. Finally, cells that are stained with both FITC and PE are sorted in bulk on a BD FACSMelody™ cell sorter.

The sorted cells are then partitioned into Gel bead in EMulsion (GEM) droplets, each derived from a single cell. Subsequently, single cell 5′ RACE-PCR is performed using the scRNA-seq microfluidics platform (10× Genomics). Briefly, the cell mix is further diluted into reagents for reverse transcription. The diluted cells are mixed with a pool of gel beads each loaded with multiple copies of an anchored 30 nucleotide oligo-dT for reverse transcription and an uniquely modified template-switching oligo which comprises, from 5′ to 3′, an universal sequencing adapter, an unique barcode of 14 nucleotides, and a randomised unique molecular identifier (UMI) of 10 nucleotides followed by a template-switching oligo for 5′ RACE. A single cell and single gel bead are encapsulated into a GEM on the microfluidic device in the water-oil surfactant interphase. Reverse transcription is carried out in each GEM so that each resulting cDNA molecule contains a UMI, the shared barcode per GEM and universal sequencing adapter at its 3′ end.

Subsequently, the emulsion is broken and all barcoded cDNA is pooled for PCR using the following primer pairs:
a) Forward primer specific for the sequencing adapter paired with reverse primer specific for mouse Ig kappa constant region;
b) Forward primer specific for the sequencing adapter paired with reverse primer specific for mouse Ig lambda constant region; and
c) Forward primer specific for the sequencing adapter paired with degenerate reverse primer specific for mouse IgG CH₁ region.

Next-generation sequencing is performed on the pooled PCR products using the primer specific for the sequencing adapter. The following sequencing information is identified:
1. a sequence encoding a heavy chain variable domain (VH) of the antibody which is (i) secreted by the corresponding plasma cell and (ii) able to bind specifically CD21; and
2. a sequence encoding a light chain variable domain (VL) of the antibody which is (i) secreted by the corresponding plasma cell and (ii) able to bind specifically CD21.

### Example 2: Screening of CD21-Specific Antibodies Using an mRNA-Labelled Goat-Anti Mouse IgG Fc

In order to generate antibodies specific to CD21 a group of three mice is immunised using an expression plasmid encoding CD21 using the genetic immunisation protocols developed by Aldevron (Aldevron Fargo, Fargo, N. Dak., USA).

The serum from individual immunised mice is evaluated for binding to purified recombinant CD21 and to CD21 expressed on cells via ELISA and flow cytometry, respectively. The spleen and bone marrow are harvested from those mice which show the highest sera conversion against CD21.

A conjugate between rat-anti-mouse CD138 monoclonal antibody (clone 281-2, BD Biosciences) and the purified recombinant CD21 is prepared using the Protein-Protein Conjugation Kit from Tri-Link Biotechnologies.

A custom synthesised polyadenylated and capped mRNA (approximately 60-100 nucleotides long) is conjugated to a goat-anti mouse IgG Fc monoclonal antibody (Thermo Fisher Cat No SA5-10227). The synthetic mRNA molecule contains a unique sequence tag.

The conjugated rat-anti mouse CD138-CD21 and the mRNA-labelled goat-anti mouse IgG Fc are mixed thoroughly into semi-solid methylcellulose-based medium (MethoCult™ M3231 from Stemcell Technologies).

The spleen and bone marrow are disrupted into a single cell suspension. Optionally, those cells with surface IgG expression, i.e. memory B cells, are depleted from the cell suspension using an excess of Dynabeads® goat-anti mouse IgG (ThermoFisher). All cells that are not bound on the beads are collected via magnetic separation where all beads and bound cells are retained on a magnet. This population of unbound cells is enriched in plasma cells.

The cell suspension is mixed gently into the modified semi-solid medium and incubated at 37° C., 5% CO₂ for between 1 and 4 hours without disturbance. During this process, the conjugated rat-anti mouse CD138-CD21 binds to CD138 on the surface of plasma cells. The conjugate bound to CD138 has the CD21 moiety free so that it can act as an antibody trap. Specifically, if a plasma cell secrets IgG specific to CD21, such secreted IgG, especially in the proximity of the corresponding cell, can be captured on the surface of the same cell. In turn, the captured IgG can bind to the mRNA-labelled goat-anti mouse IgG Fc. This leads to each antigen-specific plasma cell being labelled by the mRNA tag.

Following the incubation of the plasma cells in the modified semi-solid medium, the cells are diluted in serum free medium to reduce viscosity. The cells are then partitioned into Gel bead in EMulsion (GEM) droplets, each derived from a single cell. Subsequently, single cell 5′ RACE-PCR is performed using the scRNA-seq microfluidics platform (10× Genomics). Briefly, the cell mix is further diluted into reagents for reverse transcription. The diluted cells are mixed with a pool of gel beads each loaded with multiple copies of an anchored 30 nucleotide oligo-dT for reverse transcription and an uniquely modified template-switching oligo which comprises, from 5′ to 3′, an universal sequencing adapter, an unique barcode of 14 nucleotides, and a randomised unique molecular identifier (UMI) of 10 nucleotides followed by a template-switching oligo for 5′ RACE. A single cell and single gel bead are encapsulated into a GEM on the microfluidic device in the water-oil surfactant interphase. Reverse transcription is carried out in each GEM so that each resulting cDNA molecule contains a UMI, the shared barcode per GEM and universal sequencing adapter at its 3′ end.

Subsequently, the emulsion is broken and all barcoded cDNA is pooled for PCR using the following primer pairs:
a) Forward primer specific for the sequencing adapter paired with reverse primer specific for mouse Ig kappa constant region;
b) Forward primer specific for the sequencing adapter paired with reverse primer specific for mouse Ig lambda constant region;
c) Forward primer specific for the sequencing adapter paired with degenerate reverse primer specific for mouse IgG CH₁ region; and
d) Forward primer specific for the sequencing adapter paired with reverse primer specific for the junction between the mRNA tag and poly-A tail.

Next-generation sequencing is performed on the pooled PCR products using the primer specific for the sequencing adapter. For each plasma cell labelled by the mRNA tag, the following sequencing information is identified:
1. a sequence encoding a heavy chain variable domain (VH) of the antibody which is (i) secreted by the corresponding plasma cell and (ii) able to bind specifically CD21;
2. a sequence encoding a light chain variable domain (VL) of the antibody which is (i) secreted by the corresponding plasma cell and (ii) able to bind specifically CD21; and
3. a sequence corresponding to the mRNA tag.

### Example 3: Generating Anti-Mouse IgG, Fc-Specific Secondary Nanobody-mRNA Conjugate

Pleiner et al. (J Cell Biol, 2018, 217: 1143-54) describe nanobodies targeting the different Fc isoforms of mouse IgG. Plasmids containing the sequences encoding alpaca-anti-mouse IgG1 monoclonal dAb TP1104, alpaca-anti-mouse IgG2a monoclonal dAb TP1129, alpaca-anti-mouse IgG2a/2b monoclonal dAb TP925, and alpaca-anti-mouse IgG3 monoclonal dAb TP924 are generated. In the plasmids, each nanobody gene is flanked with two consensus regions at its 5′ and 3′ end. The 5′ consensus region comprises a T7 promoter, a short 5′ UTR derived from tobacco mosaic virus followed by a sequence encoding the FLAG tag. The 3′ consensus region comprises a sequence encoding the Gly-Gly-Gly-Ser peptide linker, a short sequence for hybridising and crosslinking with an oligo linker containing psoralen and puromycin at its 5′ and 3′, respectively, followed by a stop codon.

The generated plasmids are used as the template for in vitro transcription using mMESSAGE mMACHINE® T7 Ultra Kit from ThermoFisher. Following the in vitro transcription, an annealing reaction is set up by mixing the synthesised transcripts bearing the anti-mouse IgG nanobodies with the psoralen- and puromycin-containing oligo linker, heating to 85° C. and then slowly cooling down to room temperature. A stretch of 15 deoxyadenosine (dA15) is located in the centre of the oligo linker for purification purposes. Through the psoralen-mediated UV crosslinking, the transcripts are conjugated with the linker with the puromycin at the 3′ end.

Translation of the puromycin-containing transcripts is carried out in vitro using and Retic Lysate IVT™ Kit from ThermoFisher. During the in vitro translation, when the ribosome reaches the RNA-oligo junction and translation stalls, puromycin, which mimics the aminoacyl moiety of tRNA, enters the ribosome ‘A’ site and accepts the nascent polypeptide by forming a peptide bond. This results in the covalent linking of the newly synthesized polypeptide bearing the anti-mouse IgG nanobodies and its own mRNA, resulting in the anti-mouse IgG, Fc specific secondary nanobody-mRNA conjugate.

The anti-mouse IgG, Fc specific secondary nanobody-mRNA conjugate is purified in two steps. Firstly, it is purified via an Oligo(dT) column (ThermoFisher) to discard the proteins which have not been conjugated with mRNA. Secondly, the purified conjugates are further purified via anti-FLAG tag affinity gels (Sigma-Aldrich) to discard any free mRNA. The mRNA in the resulting nanobody-mRNA conjugate has the purpose of the mRNA tag described in Example 2.

The anti-mouse IgG, Fc specific secondary nanobody-mRNA conjugate is then used to replace the anti-mouse IgG, Fc-specific secondary antibody-mRNA conjugate in the antibody screening method described in Example 2. A suitable forward primer to amplify the mRNA tag of the nanobody-mRNA conjugate is also used.

### Example 4: Screening of CD21-Specific Antibodies Using mRNA-Labelled CD21

In order to generate antibodies specific to CD21 a group of three mice is immunised using an expression plasmid encoding CD21 using the genetic immunisation protocols developed by Aldevron (Aldevron Fargo, Fargo, N. Dak., USA).

The serum from individual immunised mice is evaluated for binding to purified recombinant CD21 and to CD21 expressed on cells via ELISA and flow cytometry, respectively. The spleen and bone marrow are harvested from those mice which show the highest sera conversion against CD21.

A conjugate between rat-anti-mouse CD138 monoclonal antibody (clone 281-2, BD Biosciences) and a goat-anti mouse IgG Fc monoclonal antibody (Thermo Fisher Cat No SA5-10227) is prepared using the Protein-Protein Conjugation Kit from Tri-Link Biotechnologies.

A custom synthesised polyadenylated and capped mRNA (approximately 60-100 nucleotides long) is conjugated to purified recombinant CD21. The synthetic mRNA molecule contains a unique sequence tag.

The conjugated rat-anti mouse CD138-goat-anti mouse IgG Fc and the mRNA-labelled CD21 are mixed thoroughly into semi-solid methylcellulose-based medium (MethoCult™ M3231 from Stemcell Technologies).

The spleen and bone marrow are disrupted into a single cell suspension. Optionally, those cells with surface IgG expression, i.e. memory B cells, are depleted from the cell suspension using an excess of Dynabeads® goat-anti mouse IgG (ThermoFisher). All cells that are not bound on the beads are collected via magnetic separation where all beads and bound cells are retained on a magnet. This population of unbound cells is enriched in plasma cells.

The cell suspension is mixed gently into the modified semi-solid medium and incubated at 37° C., 5% CO₂ for between 1 and 4 hours without disturbance. During this process, the conjugated rat-anti mouse CD138-goat-anti mouse IgG Fc binds to CD138 on the surface of plasma cells. The conjugate bound to CD138 has the goat-anti mouse IgG Fc moiety free so that it can act as an antibody trap. Specifically, if a plasma cell secrets IgG specific to goat-anti mouse IgG Fc, such secreted IgG, especially in the proximity of the corresponding cell, can be captured on the surface of the same cell. In turn, the captured IgG can bind to the mRNA-labelled CD21. This leads to each antigen-specific plasma cell being labelled by the mRNA tag.

The plasma cell population is then screened for CD21-specific antibodies following the mRNA amplification and sequence analysis method described in Example 2.

### Example 5: Generating Cell Models of Mouse Plasma Cells and Bispecific Reagents

Material and Methods

1. Establishing Two Cell Lines Bearing the Characteristics of Mouse Plasma Cells

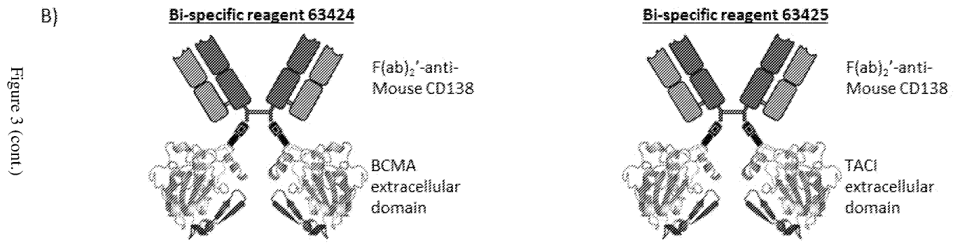

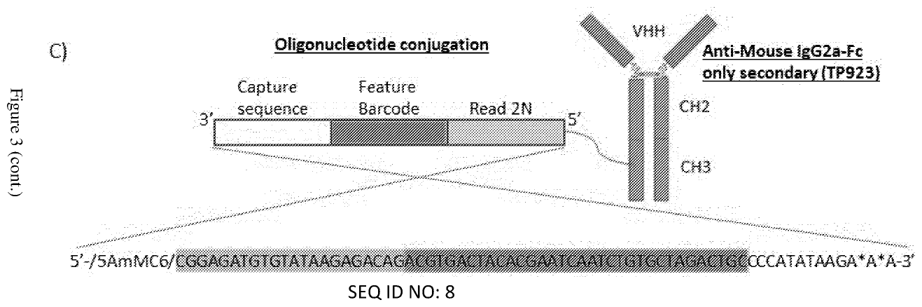

To facilitating the method development and functional testing, two cell lines bearing the characteristics of mouse plasma cells were generated. First, a human T lymphocyte cell line, Jurkat (ATCC® TIB-152™) was transduced with a retrovirus whose genome contains the full-length cDNA encoding mouse CD138 (GenBank accession number NM_011519.2) followed by the cDNA encoding monoclonal antibody 5G10, which is a mouse-anti-human BCMA having mouse IgGla, κ isotype that was discovered in-house at Autolus (London, UK). DNA encoding the 2A self-cleaving peptide was cloned before the heavy chain as well as before the light chain of 5G10 to facilitate the correct expression of all proteins (FIG. 3A). The resulting cell line, Jurkat.MuCD138.aBCMA, expresses mouse CD138 on the surface and secretes anti-BCMA antibody 5G10 (FIG. 3A).

Similarly, a human T lymphoblast cell line, Sup-T1 (ATCC® CRL-1942™) was transduced with a retrovirus whose genome contains the full-length cDNA encoding mouse CD138 followed by the cDNAs encoding 4G9, which is a mouse-anti-human TACI monoclonal antibody having mouse IgG1a, κ isotype that was discovered in-house at Autolus (London, UK). The resulting cell line, Sup-T1.MuCD138.aTACI, expresses mouse CD138 on the surface and secretes anti-TACI antibody 4G9 (FIG. 3A).

After the retroviral transduction, the mouse CD138-transduced cells were purified by staining the whole cell population with a PE-anti-mouse CD138 Antibody (BioLegend) followed with anti-PE MicroBeads (Miltenyi Biotec). The positively stained cells were isolated through magnetic cell sorting using a LS column (Miltenyi Biotec) following the manufacturer's instructions. As a result of this purification, 99% of Jurkat.MuCD138.aBCMA cells and Sup-T1.MuCD138.aTACI cells were positive for mouse CD138. Both cell lines were maintained in RPMI 1640 medium supplemented with 10% Foetal Bovine Serum (FBS) at 37° C., 5% CO₂.

2. Generating a Bispecific Reagent Comprising an Anti-Mouse CD138 Fused with a Target Antigen

The amino acid sequence of a rat-anti-mouse CD138 monoclonal antibody (Stemcell Technologies, Cat. No. 60035) was deduced using the REmAb protein sequencing platform. DNA sequences encoding the heavy and light chain variable regions (VH and Vκ, respectively) of this antibody were predicted through back-translation from the amino acid sequence and synthesised. Two constructs were generated for the expression of bispecific reagents, namely constructs 63424 and 63425. These constructs contained the rat-anti-mouse CD138 in F(ab)₂′ format fused to the extracellular domain of human BCMA or human TACI, respectively (FIG. 3B). Specifically, the constructs were designed to encode the anti-mouse CD138 Vκ region followed by the human Cκ region, a polyhistidine tag, a 2A self-cleaving peptide, the anti-mouse CD138 VH region followed by the human IgG1 CH1 region, a hinge region, and the extracellular domain of human BCMA (GenBank accession number NM_001192) or human TACI (GenBank accession number NM_012452). Bispecific reagents 63424 and 63425 were expressed in ExpiCHO-S™ Cells (ThermoFisher) transfected with the corresponding construct following the manufacturer's instructions. Four days after transfection, secreted proteins were purified from culture supernatant by affinity chromatography using the His-tag followed by size exclusion chromatography.

3. Characterising Jurkat.MuCD138.aBCMA Cells and Sup-T1.MuCD138.aTACI Cells by Staining with the Bispecific Reagents

The bispecific reagent 63424 or 63425 was added to the culture of Jurkat.MuCD138.aBCMA or Sup-T1.MuCD138.aTACI at a cell density of 2×10⁵/ml to a final concentration of 1 μg/ml. The cells were incubated at 37° C., 5% CO₂ for 1 h allowing the bispecific reagent to bind to mouse CD138 expressed on the cell surface, and to capture the corresponding antibody secreted by the cells. Subsequently, approximately 5×10⁵ cells were washed with PBS and stained for 30 min at room temperature with PE-F(ab′)₂-goat anti-mouse IgG-Fc, cross-adsorbed secondary antibody (Bethyl Laboratories), at a final concentration of 1 μg/ml. The stained cells were washed with PBS, re-suspendeded into 100 μl PBS containing 1 μM SYTOX™ Blue Dead Cell Stain (ThermoFisher), and analysed on a MACSQuant®× Flow Cytometer (Miltenyi Biotec) following the manufacturer's protocol.

4. Results:

It is difficult to isolate large quantity of plasma cells from an immunised mouse. Once isolated, the in vitro life expectancy of these cells is short. Thus, in order to facilitate the development of the method and functional testing, we have constructed two cell lines which bear the characteristics of mouse plasma cells, Jurkat.MuCD138.aBCMA and Sup-T1.MuCD138.aTACI. Both cell lines were based on malignant human T cells which were transduced with retroviral vectors to (i) stably express mouse CD138 on the surface and (ii) secrete mouse-anti-human BCMA 5G10 monoclonal antibody (MAb) and mouse-anti-human TACI 4G9 MAb, respectively.

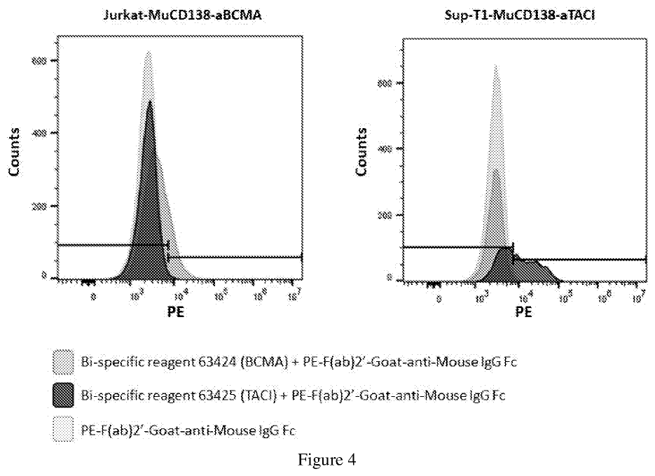

Further, bispecific reagents 63424 and 63425 formed of a monoclonal F(ab)2′-anti-mouse CD138 fused with the extracellular domain of human BCMA or human TACI, respectively, were generated. These bispecific reagents were tested by flow cytometry. Cells were incubated for 1 h with bispecific reagent 63424 or 63425, washed, and incubated with PE-labelled Goat anti-Mouse IgG-Fc secondary antibody. Results shown in FIG. 4 revealed that the shift in PE signal was detected only for the combination of Jurkat.MuCD138.aBCMA cells and bispecific reagent 63424 (left graph), and the combination of Sup-T1.MuCD138.aTACI cells and bispecific reagent 63425. This demonstrated that 63424 bound to mouse CD138 on the surface of Jurkat.MuCD138.aBCMA cells and captured the secreted anti-human BCMA 5G10 from the cells surroundings, which was then detected by the PE-labelled secondary antibody. Similarly, it is 63425 but not 63424 that bound to mouse CD138 on the surface of Sup-T1.MuCD138.aTACI cells and captured the secreted anti-Human TACI 4G9 from the surroundings, which was then detected by the PE-labelled secondary antibody.

Differences in the levels of foreign protein expression existed in Jurkat vs Sup-T1 cells. We noticed that the expression of mouse CD138 on the surface of Jurkat cells was 5-fold lower in average than that on Sup-T1 cells (data not shown). The level of secreted antibodies from Jurkat cells might also be significantly lower than that from Sup-T1 cells. This may explain the lower fluorescence signal as observed from (Jurkat.MuCD138.aBCMA) cells than (Sup-T1.MuCD138.aTACI) cells as shown in FIG. 4.

These data validate not only the purposefully designed features of both mouse plasma cell-like cell lines, but also the specificity and function of the two bispecific reagents generated for this work.

### Example 6: Generating Anti-Mouse IgG2a Fc-Specific Secondary Antibody Conjugated to an Oligonucleotide

DNA encoding the variable region of an alpaca-anti-Mouse IgG2a, Fc-specific nanobody, i.e. TP923 (Pleiner et al., 2018, J Cell Biol 217:1143-54) was synthesised and subcloned immediately upstream of the sequence encoding human IgG1 hinge, CH2 and CH3 in an expression vector, such that the resulting DNA construct was in a heavy chain only antibody format (FIG. 3C). The resulting secondary antibody was expressed in ExpiCHO-S™ Cells and purified using a HiTrap® Protein A High Performance column (Sigma-Aldrich) following the manufacturer's instructions.

A 69-base oligonucleotide was designed following the guidance of antibody-oligonucleotide conjugation published by 10×Genomics (Protocol number CG000149), synthesised and purified was custom synthesized and purified via HPLC by IDT. The sequence of this oligonucleotide is shown as SEQ ID NO: 8 and in FIG. 3C, where the unique specific tag sequence is highlighted. The TP923 secondary antibody was conjugated with the synthesized oligonucleotide using the Thunder-Link® PLUS Oligo Conjugation System (Expedeon), following the manufacturer's instructions.

### Example 7: Screening of BCMA- and TACI-Specific Antibodies Using mRNA-Labelled Anti-Mouse IgG2a Fc-Specific Antibody

### 1. Experiment I—Co-Culturing Jurkat.MuCD138.aBCMA Cells and Sup-T1.MuCD138.aTACI Cells with the Bispecific Reagent 63424 and the Oligo-Conjugated Secondary Antibody

Jurkat.MuCD138.aBCMA cells and Sup-T1.MuCD138.aTACI cells were washed separately in RPMI 1640 medium, re-suspended into RPMI 1640 supplemented with 10% FBS to 1×10⁶ cells/ml and kept on ice. Then, 5×10⁵ Jurkat.MuCD138.aBCMA and 1.5×10⁶ Sup-T1.MuCD138.aTACI cells were combined, pelleted, and the cell pellet was gently re-suspended into 2 ml of freshly prepared RPMI 1640 supplemented with 10% FBS, 2.5 μg/ml bispecific reagent 63424 containing the BCMA extracellular domain, and oligo-conjugated secondary antibody TP923. The re-suspended cells were immediately transferred into a well on a 6-well tissue culture plate. The plate was incubated for 4 h at 37° C., 5% CO₂. Subsequently, the cells were washed three times with ice-cold RPMI 1640, and re-suspended into 1.5 ml ice-cold RPMI 1640. The final cell count was 7.8×10⁵ cells/ml.

### 2. Experiment II—Co-Culturing Jurkat.MuCD138.aBCMA Cells and Sup-T1.MuCD138.aTACI Cells with the Bispecific Reagent 63425 and the Oligo-Conjugated Secondary Antibody

Jurkat.MuCD138.aBCMA cells and Sup-T1.MuCD138.aTACI cells were washed separately

in RPMI 1640 medium, re-suspended into RPMI 1640 supplemented with 10% FBS to 1×10⁶ cells/ml and kept on ice. Then, 5×10⁵ Jurkat.MuCD138.aBCMA and 1.5×10⁶ Sup-T1.MuCD138.aTACI cells were combined, pelleted, and the cell pellet was gently re-suspended into 2 ml of freshly prepared RPMI 1640 supplemented with 10% FBS, 2.5 μg/ml bispecific reagent 63425 containing the TACI extracellular domain, and oligo-conjugated secondary antibody The re-suspended cells were immediately transferred into a well on a 6-well tissue culture plate. The plate was incubated for 4 h at 37° C., 5% CO₂. Subsequently, the cells were washed three times with ice-cold RPMI 1640, and re-suspended into 1.5 ml ice-cold RPMI 1640. The final cell count was 1×10⁶ cells/ml.

### 3. Partition of Cells from Experiments I and II on the 10×Genomics Platform, Reverse Transcription and Sequencing

Chromium Single Cell 5′ Library & Gel Bead Kit and Chromium Single Cell 5′ Feature Barcode Library Kit (10×Genomics) were used for the following processes: single cell partitioning into Gel Beads-in-emulsion (GEMs), simultaneous synthesis of 10× barcoded cDNA and extension of the oligonucleotide having the specific tag sequence in individual GEMs, and construction of Illumina sequencing libraries. All of these processes were carried our following the manufacturer's instructions.

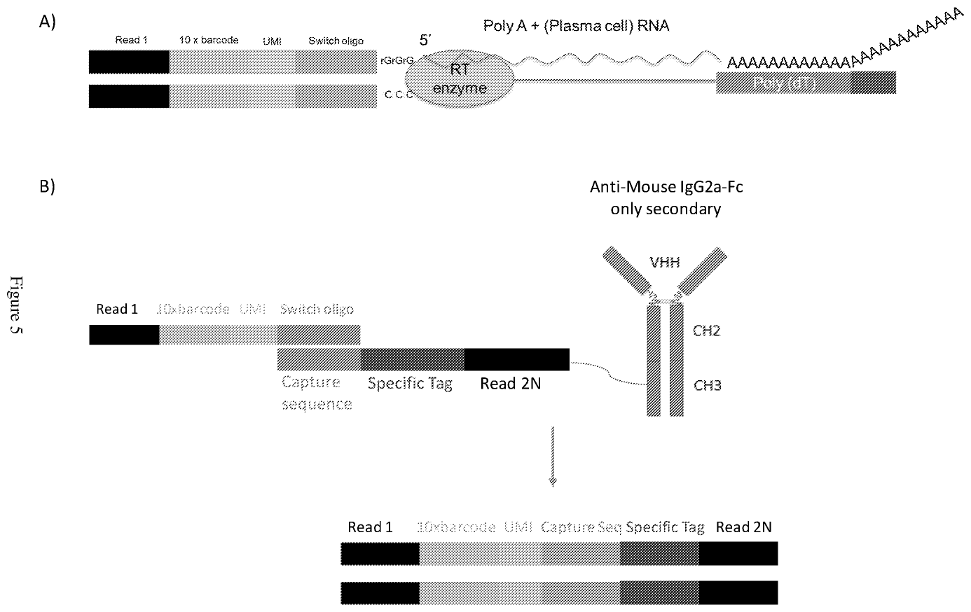

Briefly, 1,700 cells obtained in Experiments I or II, i.e. cells labelled with bispecific reagents and oligo-conjugated secondary antibodies with the specific tag sequence, were loaded onto separate wells on a Chromium Chip A. Controls without oligo-conjugated secondary antibodies were also added. The chip was run on a Chromium Controller for GEM formation. were further diluted into reagents for reverse transcription, which included 30 nucleotide oligo-dT and reverse transcriptase. The diluted cell and reverse transcription reaction mix were mixed with a pool of gel beads each anchored with a unique modified template-switching oligo which comprised, from 5′ to 3′, a sequencing adapter, a unique barcode of 16 nucleotides (10× Barcode), a randomised unique molecular identifier (UMI) of 10 nucleotides, followed by a template switching oligo of 13 nucleotides. A single cell and a single gel bead were encapsulated into a GEM on a microfluidic device at the water-oil surfactant interface. Reverse transcription was carried out in each GEM so that each resulting cDNA molecule contained the sequencing adapter, a UMI, and a shared 10× barcode per GEM at its 5′ end (FIG. 5A). Simultaneously, if this cell was labelled with oligo-conjugated secondary antibodies as described in Example 6 (above), this DNA sequence was also barcoded at its 5′ end with the sequencing adapter, a UMI, and a shared 10× barcode per GEM (FIG. 5B).

Subsequently, the GEMs were dissolved, and all cDNA molecules were purified with the Dynabeads provided by the kits. For each experiment, all the 10× barcoded cDNAs were pooled, amplified and purified according to their size.

Two Illumina sequencing libraries were constructed for each experiment: (i) a 5′ Gene Expression (GEX) library derived from the 10× barcoded cDNAs, which contains Read 1 and Read 2 Illumina sequencing priming sites and which has an average fragment length of 420 bp; and (ii) a library derived from the oligonucleotide having the specific tag sequence corresponding to the secondary antibody, which contains Read 1 and Read 2N Illumina sequencing priming sites and which has an average fragment length of 200 bp. Multiplexing sequencing was enabled by adding a unique sample index to the 3′ end of each library.

Finally, libraries (i) and (ii) of Experiments I and II were mixed together for sequencing. Briefly, 32 nM of the 5′ GEX library obtained from the cells of Experiment I, 4 nM of the corresponding specific tag sequence library from Experiment I, 32 nM of the 5′ GEX library obtained from the cells of Experiment II, and 4 nM of the corresponding specific tag sequence library from Experiment II were mixed together in a total volume of 50 μl. This multiplexed library was sequenced on one lane on an Illumina HiSeq 4000 system with the read length set at 2×150 bp paired-end (Genewiz, South Plainfield, N.J., USA).

### 4. Sequencing Data Analysis

The bulk sequencing data was analysed using 10×Genomics software package Cell Ranger version 3.1 on a Linux server. Cell Ranger is a set of analysis pipelines that process the RNA-seq output to align reads, generate feature-barcode matrices based on detected 10×Barcodes associated with single cells and perform clustering and gene expression analysis.

Briefly, the bulk sequencing data was demultiplexed by Cell Ranger ‘mkfastq’ pipeline according to the sample index associated with individual sequencing library. This yielded four FASTQ files, two for libraries (i) and (ii) obtained from Experiment I and two for libraries (i) and (ii) obtained from Experiment II. Subsequently, the Cell Ranger ‘count’ pipeline was used to open and analyse individual FASTQ files.

For the current experiment using Jurkat.MuCD138.aBCMA cells and Sup-T1.MuCD138.aTACI cells, the transcripts encoding the anti-BCMA or anti-TACI antibodies are located downstream of Mouse CD138 transcript in their viral transgene, and therefore they are absent in the sequencing reads of the 5′ GEX library since the reads have a short average length (FIG. 3A). Nevertheless, both cell types can be differentiated by their unique gene expression profiles. For example, Jurkat cells express a functional TCR in which the a subunit is encoded by rearranged TRAV8-4-TRAJ3, and the f3 subunit is encoded by rearranged TRBV12-3-TRBD1-TRBJ1-2. In contrast, Sup-T1 cells do no express any functional TCR. Additionally, Sup-T1 cells express both CD4 and CD8 strongly, whereas Jurkat cells express CD4 weakly and does not express CD8.

The ‘count’ pipeline performed cell clustering based on the gene expression profile of individual cells and facilitated assigning an identity, i.e. Jurkat.MuCD138.aBCMA or Sup-T1.MuCD138.aTACI, to each single cell. Furthermore, the unique 10× Barcode associated with cDNA from each single cell and the specific tag sequence from the secondary antibody were also identified by the ‘count’ pipeline. Subsequently, matching individual 10× Barcodes corresponding to specific tag sequence from the secondary antibody with Jurkat.MuCD138.aBCMA or Sup-T1.MuCD138.aTACI cells was done in Microsoft Excel.

The following sequencing information is identified for each cell:
1. VH sequence with a 10× barcode,
2. VL sequence with the same 10× barcode, and
3. the specific sequence tag with the same 10× barcode.

The presence of these three sequences in the resulting sequencing data confirms that the antibody secreted by the cell binds to the target antigen of the bispecific reagent.

1. A bispecific reagent which comprises:
(i) a first domain which binds specifically to an antigen expressed by an antibody-producing cell; and
(ii) a second domain which comprises a target antigen.

2. The bispecific reagent according to claim 1, wherein the antigen expressed by the antibody-producing cell is selected from CD138, CD38, CD98, Sca-1, Ly6c1/2, Ly6k, CD28, transmembrane activator and calcium modulator and cyclophilin ligand interactor (TACI), B-cell maturation antigen (BCMA), and SLAM7.

3. The bispecific reagent according to any of claim 1 or 2, wherein the first domain is selected from a monoclonal antibody, a fragment thereof, and a domain antibody (dAb).

4. A complex C1 which comprises:
a) an antibody-producing cell; and
b) a bispecific reagent according to any of claims 1 to 3, which is bound to the antibody-producing cell via the antigen expressed by the antibody-producing cell.

5. A complex C2 which comprises:
a) an antibody-producing cell;
b) a bispecific reagent according to any of claims 1 to 3, bound to the antibody-producing cell via the antigen expressed by the antibody-producing cell; and
c) an antibody which:
(i) is secreted by the antibody-producing cell, and
(ii) specifically binds the target antigen,
wherein the antibody is bound to the bispecific reagent via the target antigen.

6. A detection agent which comprises an anti-IgG antibody which is labelled with a nucleic acid which comprises a specific tag sequence.

7. A detection agent according to claim 7, wherein the anti-IgG antibody is a scFv or a nanobody.

8. A detection agent according to claim 7, wherein the nanobody is selected from Alpaca-anti-Mouse IgG1 monoclonal nanobody TP1104, Alpaca-anti-Mouse IgG2a monoclonal nanobody TP1129, Alpaca-anti-Mouse IgG2a/2b monoclonal nanobody TP925, Alpaca-anti-Mouse IgG3 monoclonal nanobody TP924, and alpaca-anti-mouse IgG2a Fc monoclonal nanobody TP923.

9. A detection agent according to any of claims 6 to 8, wherein the nucleic acid which comprises a specific tag sequence is DNA.

10. A detection agent according to claim 9, wherein the nucleic acid which comprises a specific tag sequence further comprises a sequence suitable for priming in next generation sequencing (NG sequencing).

11. A detection agent according to any of claim 9 or 10, wherein the nucleic acid which comprises a specific tag sequence further comprises a sequence that is complementary to that of a template switching oligonucleotide.

12. A detection agent according to any of claims 9 to 11, wherein the nucleic acid comprises the sequence shown as SEQ ID NO: 8.

13. A detection agent according to any of claims 6 to 8, wherein the nucleic acid which comprises a specific tag sequence is RNA.

14. A detection agent according to claim 13, wherein the nucleic acid which comprises a specific tag sequence further comprises a specific tag sequence and a Poly(A) tail.

15. A detection agent according to any of claim 13 or 14, wherein the RNA sequence encodes the anti-IgG antibody.

16. A detection agent according to any of claims 13 to 15, wherein the anti-IgG antibody and the RNA sequence are linked together via a puromycin.

17. A detection agent according to any of claims 13 to 16, wherein the RNA sequence comprises an RNA analogue.

18. A detection agent according to any of claims 13 to 17, wherein the RNA sequence is at least 450 nucleotides long.

19. A complex C3 which comprises:
a) an antibody-producing cell;
b) a bispecific reagent according to any of claims 1 to 3, bound to the antibody-producing cell via the antigen expressed by the antibody-producing cell;
c) an antibody which:
(i) is secreted by the antibody-producing cell, and
(ii) specifically binds the target antigen,
wherein the antibody is bound to the bispecific reagent via the target antigen; and
d) a detection agent according to any of claims 6 to 18, bound to the antibody of (c).

20. An assay for identifying an antibody-producing cell which produces an antibody which binds specifically to a target antigen, which comprises the following steps:
(i) providing a population of antibody-producing cells;
(ii) binding a bispecific reagent according to any of claims 1 to 3 to the cells;
(iii) incubating the cells from step (ii) with a target antigen;
(iv) adding a detection agent according to any of claims 6 to 18 to the cells from step (iii);
(v) partitioning the cells from (iv) into partitions, wherein each partition contains a single cell and a unique barcode molecule;
(vi) performing reverse transcription such that all RNA sequences in the cell within the partition and the RNA sequence of the detection reagent (if present) are barcoded with the unique barcode molecule;
(vii) disrupting the partitions and pooling the barcoded nucleic acid sequences from (vi)
(viii) analysing the pooled sequences to find sets of sequences with the same unique barcode which comprise:
(a) a sequence encoding a heavy chain variable domain (VH);
(b) a sequence encoding a light chain variable domain (VL); and
(c) a sequence corresponding to the reverse transcript of the RNA sequence of the detection agent

21. An assay according to claim 20, which comprises a step of sorting cells prior to step (i).

22. An assay according to any of claim 20 or 21, wherein the reverse transcription is performed using an oligonucleotide which is complementary to a sequence encoding the IgG heavy chain constant region.

23. An assay according to any of claims 20 to 22, wherein the reverse transcription is performed using an oligonucleotide which is complementary to a sequence encoding the IgG light chain constant region.

24. An assay according to any of claims 20 to 23, wherein a step of DNA amplification is performed after step after step (vi) and prior to step (vii).

25. An assay according to any of claims 20 to 24, wherein a step of DNA amplification is performed after step after step (vii) and prior step (viii).

26. An assay according to any of claims 20 to 25, wherein the analysis of step (viii) comprises a step of DNA sequencing.

27. A kit for use in the assay according to any of claims 20 to 26, which comprises the bispecific reagent according to any of claims 1 to 3 and the detection agent according to claims 6 to 18.

28. The kit according to claim 27, further comprising one or more components selected from the group consisting of partitioning fluids, barcode molecule libraries, which may be associated or not with beads (e.g. microcapsules), reagents for disrupting cells, reagents for amplifying nucleic acids, and any other component required to carry out the assay of the invention.

29. The kit according to any of claim 27 or 28, further comprising instructions for using the kit according to the assay according to any of claims 20 to 26.

30. A bispecific reagent which comprises:
(i) a first domain which binds specifically to an antigen expressed by an antibody-producing cell; and
(ii) a second domain which comprises a binding domain which binds specifically to IgG.

31. The bispecific reagent according to claim 30, wherein the antigen expressed by the antibody-producing cell is selected from CD138, CD38, CD98, Sca-1, Ly6c1/2, Ly6k, CD28, transmembrane activator and calcium modulator and cyclophilin ligand interactor (TACI), B-cell maturation antigen (BCMA), and SLAM7.

32. The bispecific reagent according to any of claim 30 or 31, wherein the first domain is selected from a monoclonal antibody, a fragment thereof, and a nanobody or dAb.

33. The bispecific reagent according to any of claims 30 to 32, wherein the nanobody is selected from Alpaca-anti-Mouse IgG1 monoclonal nanobody TP1104, Alpaca-anti-Mouse IgG2a monoclonal nanobody TP1129, Alpaca-anti-Mouse IgG2a/2b monoclonal nanobody TP925, Alpaca-anti-Mouse IgG3 monoclonal nanobody TP924, and alpaca-anti-mouse IgG2a Fc monoclonal nanobody TP923.

34. A complex D1 which comprises:
a) an antibody-producing cell; and
b) a bispecific reagent according to any of claims 30 to 33, which is bound to the antibody-producing cell via the antigen expressed by the antibody-producing cell.

35. A complex D2 which comprises:
a) an antibody-producing cell;
b) a bispecific reagent according to any of claims 30 to 33, which is bound to the antibody-producing cell via the antigen expressed by the antibody-producing cell; and
c) an antibody which:
(i) is secreted by the antibody-producing cell, and
(ii) specifically binds the target antigen,
wherein the antibody is bound to the bispecific reagent via the binding domain which binds specifically to IgG.

36. A detection agent which comprises the target antigen which is labelled with a nucleic acid which comprises a specific tag sequence.

37. A detection agent according to claim 36, wherein the nucleic acid which comprises a specific tag sequence is DNA.

38. A detection agent according to claim 37, wherein the nucleic acid which comprises a specific tag sequence further comprises a sequence suitable for priming in NG sequencing.

39. A detection agent according to any of claim 37 or 38, wherein the nucleic acid which comprises a specific tag sequence further comprises a sequence that is complementary to that of a template switching oligonucleotide.

40. A detection agent according to any of claims 37 to 39, wherein the nucleic acid comprises the sequence shown as SEQ ID NO: 8.

41. A detection agent according to claim 36, wherein the nucleic acid which comprises a specific tag sequence is RNA.

42. A detection agent according to claim 41, wherein the nucleic acid which comprises a specific tag sequence further comprises a specific tag sequence and a Poly(A) tail.

43. A detection agent according to any of claim 41 or 42, wherein the RNA sequence encodes the target antigen.

44. A detection agent according to any of claims 41 to 43, wherein the target antigen and the RNA sequence are linked together via a puromycin.

45. A detection agent according to any of claims 30 to 35, wherein the RNA sequence is at least 450 nucleotides long.

46. A complex D3 which comprises:
a) an antibody-producing cell;
b) a bispecific reagent according to any of claims 30 to 33, which is bound to the antibody-producing cell via the antigen expressed by the antibody-producing cell; and
c) an antibody which:
(i) is secreted by the antibody-producing cell, and
(ii) specifically binds the target antigen,
wherein the antibody is bound to the bispecific reagent via the binding domain which binds specifically to IgG; and
d) a detection agent according to any of claims 36 to 45, bound to the antibody of (c).

47. An assay for identifying an antibody-producing cell which produces an antibody which binds to a target antigen, which comprises the following steps:
(i) providing a population of antibody-producing cells;
(ii) binding a bispecific reagent according to any of claims 30 to 33 to the cells;
(iii) incubating the cells from step (ii) with a target antigen;
(iv) adding a detection agent according to any of claims 36 to 45 to the cells from step (iii);
(v) partitioning the cells from (iv) into partitions, wherein each partition contains a single cell and a unique barcode molecule;
(vi) performing reverse transcription such that all RNA molecules in the cell within the partition and the RNA sequence of the detection reagent (if present) are barcoded with the unique barcode molecule;
(vii) disrupting the partitions and pooling the barcoded nucleic acid sequences from (vi)
(viii) analysing the pooled sequences to find sets of sequences with the same unique barcode which comprise:
(a) a sequence encoding a heavy chain variable domain (VH);
(b) a sequence encoding a light chain variable domain (VL); and
(c) a sequence corresponding to the reverse transcript of the RNA sequence of the detection agent

48. An assay according to claim 47, which comprises a step of sorting cells prior to step (i).

49. An assay according to any of claim 47 or 48, wherein the reverse transcription is performed using an oligonucleotide which is complementary to a sequence encoding the IgG heavy chain constant region.

50. An assay according to any of claims 47 to 49, wherein the reverse transcription is performed using an oligonucleotide which is complementary to a sequence encoding the IgG light chain constant region.

51. An assay according to any of claims 47 to 50, wherein a step of DNA amplification is performed after step after step (vi) and prior to step (vii).

52. An assay according to any of claims 47 to 51, wherein a step of DNA amplification is performed after step after step (vii) and prior step (viii).

53. An assay according to any of claims 47 to 52, wherein the analysis of step (viii) comprises a step of DNA sequencing.

54. A kit for use in the assay according to any of claims 47 to 53, which comprises the bispecific reagent according to claims 30 to 33 and the detection agent according to claims 36 to 45.

55. The kit according to claim 54, further comprising one or more components selected from the group consisting of partitioning fluids, barcode molecule libraries, which may be associated or not with beads (e.g. microcapsules), reagents for disrupting cells, reagents for amplifying nucleic acids, and any other component required to carry out the assay of the invention.

56. The kit according to any of claim 54 or 55, further comprising instructions for using the kit according to the assay according to any of claims 47 to 59.


SEQUENCE LISTING (10 sequences)

SEQ ID NO: 1 - RNA, 55 nt, Artificial Sequence
  unique sequence segment (USS)
ucaccccucaacaacuagcaaaggcagccccauaaacacacaguauguuuuuuga

SEQ ID NO: 2 - RNA, 56 nt, Artificial Sequence
  unique sequence segment (USS)
uaggaaaguuggucuucgccaucauggcaguugcuugcaauguaauuuucaguuaa

SEQ ID NO: 3 - RNA, 75 nt, Artificial Sequence
  specific RNA tag sequence
ucaccccucaacaacuagcaaaggcagccccauaaacacacaguauguuuuuugaaaaaaaaaaaaaaaaaaaaa

SEQ ID NO: 4 - RNA, 76 nt, Artificial Sequence
  specific RNA tag sequence
uaggaaaguuggucuucgccaucauggcaguugcuugcaauguaauuuucaguuaaaaaaaaaaaaaaaaaaaaaa

SEQ ID NO: 5 - DNA, 34 bp, Artificial Sequence
  unique sequence segment (USS)
acgtgactacacgaatcaatctgtgctagactgc

SEQ ID NO: 6 - DNA, 22 bp, Artificial Sequence
  Read 2N adapter sequence
cggagatgtgtataagagacag

SEQ ID NO: 7 - DNA, 13 bp, Artificial Sequence
  sequence complementary to a template switching oligonucleotide
cccatataagaaa

SEQ ID NO: 8 - DNA, 69 bp, Artificial Sequence
  specific tag sequence
cggagatgtgtataagagacagacgtgactacacgaatcaatctgtgctagactgccccatataagaaa

SEQ ID NO: 9 - protein, 4 aa, Artificial Sequence
  peptide linker sequence
GGGS

SEQ ID NO: 10 - RNA, 24 nt, Artificial Sequence
  poly (A) tail
aaaaaaaaaaaaaaaaaaaaaaaa